SEQUENCE LISTING (12 sequences)

SEQ ID NO: 1 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 2 - DNA, 566 bp, Homo sapiens
actctccctgcacagctcagcgggacctcagccatgagacttctcatcctggccctccttggcatctgctctctcactgcatacattgtggaaggtgtagggagtgaagtctcacataggaggacctgtgtgagcctcactacccagcgactgccagttagcagaatcaagacctacaccatcacggaaggctccttgagagcagtaatttttattaccaaacgtggcctaaaagtctgtgctgatccacaagccacgtgggtgagagacgtggtcaggagcatggacaggaaatccaacaccagaaataacatgatccagaccaagccaacaggaacccagcaatcgaccaatacagctgtgaccctgactggctagtagtctctggcaccctgtccgtctccagccagccagctcatttcactttacaccctcatggactgagattatactcaccttttatgaaagcactgcatgaataaaattattcctttgtatttttacttttaaatgtcttctgtattcacttatatgttctaattaataaattatttattattaagaaa

SEQ ID NO: 3 - DNA, 500 bp, Homo sapiens
cacagctgggttctgaagcttctgagttctgcagcctcacctctgagaaaacctcttttccaccaataccatgaagctctgcgtgactgtcctgtctctcctcatgctagtagctgccttctgctctccagcgctctcagcaccaatgggctcagaccctcccaccgcctgctgcttttcttacaccgcgaggaagcttcctcgcaactttgtggtagattactatgagaccagcagcctctgctcccagccagctgtggtattccaaaccaaaagaagcaagcaagtctgtgctgatcccagtgagacctgggtccaggagtacgtgtatgacctggaactgaactgagctgctcagagacaggaagtcttcagggaaggtcacctgagcccggatgcttctccatgagacacatctcctccatactcaggactcctctccgcagttcctgtcccttctcttaatttaatcttttttatgtgccgtgttattgtattag

SEQ ID NO: 4 - DNA, 1641 bp, Homo sapiens
  misc_feature (150)..(150): n is a, c, g, or t
gatgcagcgtagctcttagggggaggtgtgagtccttggaaagctcacaagaatttcagaggggcccgagatgcgagtcggactcccttccgaggcgtcgtcttgcccatggtcagcgcgggggccgcgggacccgccagagcgcaggcnggagtgggctggcgcacgtggtagcgagggagttgggtctccaggttgtgcgaggagcaaatgatgaccgccaaggccgtagacaaaatcccagtaactctcagtggttttgtgcaccagctgtctgacaacatctacccggtggaggacctcgccgccacgtcggtgaccatctttcccaatgccgaactgggaggcccctttgaccagatgaacggagtggccggagatggcatgatcaacattgacatgactggagagaagaggtcgttggatctcccatatcccagcagctttgctcccgtctctgcacctagaaaccagaccttcacttacatgggcaagttctccattgaccctcagtaccctggtgccagctgctacccagaaggcataatcaatattgtgagtgcaggcatcttgcaaggggtcacttccccagcttcaaccacagcctcatccagcgtcacctctgcctcccccaacccactggccacaggacccctgggtgtgtgcaccatgtcccagacccagcctgacctggatcacctgtactctccgccaccgcctcctcctccttattctggctgtgcaggagacctctaccaggacccttctgcgttcctgtcagcagccaccacctccacctcttcctctctggcctacccaccacctccttcctatccatcccccaagccagccacggacccaggtctcttcccaatgatcccagactatcctggattctttccatctcagtgccagagagacctacatggtacagctggcccagaccgtaagccctttccctgcccactggacaccctgcgggtgccccctccactcactccactctctacaatccgtaactttaccctggggggccccagtgctggggtgaccggaccaggggccagtggaggcagcgagggaccccggctgcctggtagcagctcagcagcagcagcagccgccgccgccgccgcctataacccacaccacctgccactgcggcccattctgaggcctcgcaagtaccccaacagacccagcaagacgccggtgcacgagaggccctacccgtgcccagcagaaggctgcgaccggcggttctcccgctctgacgagctgacacggcacatccgaatccacactgggcataagcccttccagtgtcggatctgcatgcgcaacttcagccgcagtgaccacctcaccacccatatccgcacccacaccggtgagaagcccttcgcctgtgactactgtggccgaaagtttgcccggagtgatgagaggaagcgccacaccaagatccacctgagacagaaagagcggaaaagcagtgccccctctgcatcggtgccagccccctctacagcctcctgctctgggggcgtgcagcctgggggtaccctgtgcagcagtaacagcagcagtcttggcggagggccgctcgccccttgctcctctcggacccggacaccttga

SEQ ID NO: 5 - DNA, 5635 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

SEQ ID NO: 6 - DNA, 674 bp, Homo sapiens
acaggacacagctaggttctgaagcttctgagttctgcagcctcacctctgagaaaacctctttgccaccaataccatgaagctctgcgtgactgtcctgtctctcctcgtgctagtagctgccttctgctctctagcactctcagcaccaatgggctcagaccctcccaccgcctgctgcttttcttacaccgcgaggaagcttcctcgcaactttgtggtagattactatgagaccagcagcctctgctcccagccagctgtggtattccaaaccaaaagaggcaagcaagtctgcgctgaccccagtgagtcctgggtccaggagtacgtgtatgacctggaactgaactgagctgctcagagacaggaagtcttcagggaaggtcacctgagcctggatgcttctccatgagccgcatctcctccatactcaggactcctctccgcagttcctgtctcttctcttaatgtaatctcttttatgtgctgtattattgtattaggtgttatttccattatttatattagtttagccaaaggataagtgtcccctatggggatggtccactctcactctttctctgctgttgcaaatacatggataacaccgttaattccatgtgttttcataataaaactttaaaataaaatgcaaaaaaaaaaaaaaaaaa

SEQ ID NO: 7 - DNA, 1559 bp, Homo sapiens
ccccagtctgagaacaagaaagaagaacttctgtctcgagggtctcactgtcaaccaggccagagtgcagtgaagatcatacctcactacatccgtgaactcccgggctcctcccacctaagtctcttgagtagctgggacttcaggagactgaagccaaggataccagcagagccaacatttgcttcaagttcctgggcctgctgacagcgtgcaggatgctgttggaacccggcagaggctgctgtgccctggccatcctgctggcaattgtggacatccagtctggtggatgcattaacatcaccagctcagcttcccaggaaggaacgcgactaaacttaatctgtactgtatggcataagaaagaagaggctgaggggtttgtagtgtttttgtgcaaggacaggtctggagactgttctcctgagaccagtttaaaacagctgagacttaaaagggatcctgggatagatggtgttggtgaaatatcatctcagttgatgttcaccataagccaagtcacaccgttgcacagtgggacctaccagtgttgtgccagaagccagaagtcaggtatccgccttcagggccattttttctccattctattcacagagacagggaactacacagtgacgggattgaaacaaagacaacaccttgagttcagccataatgaaggcactctcagttcaggcttcctacaagaaaaggtctgggtaatgctggtcaccagccttgtggcccttcaagctttgtaagccttgtgccaaaagaaacttttaaaacagctacagcaagatgagtctgactatggcttagtatctttctcattacaataggcacagagaagaatgcaacagggcacaggggaagagatgctaaatataccaagaatctgtggaaatataagctggggcaaatcagtgtaatccttgactttgctcctcaccatcagggcaaacttgccttcttccctcctaagctccagtaaataaacagaacagctttcaccaaagtgggtagtatagtcctcaaatatcggataaatatatgcgtttttgtaccccagaaaaacttttcctccctcttcatcaacatagtaaaataagtcaaacaaaatgagaacaccaaattttgggggaataaatttttatttaacactgcaaaggaaagagagagaaaacaagcaaagataggtaggacagaaaggaagacagccagatccagtgattgacttggcatgaaaatgagaaaatgcagacagacctcaacattcaacaacatccatacagcactgctggaggaagaggaagatttgtgcagaccaagagcaccacagactacaactgcccagcttcatctaaatacttgttaacctctttggtcatttctctttttaaataaatgcccatagcagtatttggagtattttcttttctcctaaatccacaaactctcttctttctctttggacagatgacctcttgtcatagttaagcagagagtgggcaggatattcctgataggaggaactacatgaataaaggggtaag

SEQ ID NO: 8 - DNA, 813 bp, Homo sapiens
agctggtttcagacttcagaaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaaaaaaaaaaaaaaaaaa

SEQ ID NO: 9 - DNA, 2422 bp, Homo sapiens
atgtggtggagagttctcagcttgctggcatggttccccttgcaagaggcctctctgactaaccacacagaaaccatcaccgtggaggaaggccagacgctcactctaaagtgtgtcacttctctgaggaagaactcctccctccagtggctgaccccctcagggttcaccatttttttaaatgagtatcctgctttaaaaaattccaaataccagcttcttcatcactcggccaatcagctctccatcactgtgcctaacgtaaccctgcaagatgaaggcgtgtacaagtgcttacattacagcgactctgtaagcacaaaggaagtgaaagtgattgtgctggcaactcctttcaagccaatcctggaagcttcagttatcagaaagcaaaatggagaagaacatgttgtactcatgtgctccaccatgagaagcaagccccctccgcagataacctggctacttgggaatagcatggaagtgtccggtggaacgctccatgaatttgaaactgatgggaagaaatgtaatactaccagcactctcataatccacacttatggcaaaaattcaacggtggactgcattatccgacacagaggcctgcaagggagaaaactagtagcacccttccggtttgaagatttggttactgatgaagagacagcttcagatgctctggagagaaactctctatcctctcaagacccacagcagcccaccagtactgtctcagtaacggaagattctagtacatcggagattgacaaggaagagaaagaacaaaccactcaagatcctgacttgaccaccgaagcaaatcctcagtatttaggactggcaagaaagaaaagtggcatcctgctgctcacgctggtgtccttcctcattttcatactcttcatcatagtccagctcttcatcatgaagctgaggaaagcacatgtgatatggaagaaagaaaacgaagtttcagaacacacactagaaagttacagatcaaggtcaaataatgaagaaacatcatctgaagagaaaaatggccaatcttcccaccctatgcgttgcatgaactacatcacaaagttgtactcagaagcaaaaacaaagaggaaggaaaatgtacaacattcaaaattagaagaaaagcacatccaagtaccagagagtattgtgtagtgctctctgcaatggaacatgtgatttcagggttgccgcagtgtcacctcagtggaccagcctgggggaaggagcttaattgctgagacattaataatgacctcttagtgcaatgcaagatggtgtcctcggataatgatctgccccggagctagggcagcaacatgaggaccaaaccatgcacataaagcttgtagtttaaaaaagaaaagcaaaaaaataattatgcctgacactacttcagagcaggaggattctacgaagccttggggatcagggtcagtgtgagcagctaacatcctacctcaaatggaacaggattttttgatgctttgctctaatggaactgttttaaaaatttttttttctttttaatattttcttctggtcacaaaataaagaaatttgggatgcaaagtacctaaagatctctgatcctaagaagttacttctggccaggcgcggtggctcatgcctgtaatcctagcactttgggaggctgaggtaggcagatcacttgaggtcaggagttggagaccagcctggccaacatagtgaaaccccgtctctactaaaaatgcaaaaattagccaggcgtagtggtgcgcacctgtagtctcagatacttgggaggctgagggtggagaatcgcttgaacctgggaggtggagattgcagtgagtcaagatctcaccactgcactccagcctgggcgacagagggagactctgtctcaaaaaaaaagaagttacttccaagacagacttttaacatgtaaccagcacaaagcaatgtcagggaggagtgatacatgacaataaatgagtcagatgagcaagaaggccccagaacccatgccccaaggcacaaagaggagctcagggtaggcccaaagattcgaattccttagattactaaatcacatgagcggaccttgtctgtcagataagatttttacctggaaattccatgaccaatacatgtgcaaaagaaaatgatgggttgaatttactgattattgacctaatggatggctttttaaaatgttttaataaaaagcagaatagatgtttgtttttctagtggttataccaagttatacttcctgttttcacgtgtgaaagtaacatgggacatgcctttcttttccgatcagtttatttaagctatacagcaaactttggcatttatgtggagcatttctcattgttggaatctgaataaaccaattacaaaataa

SEQ ID NO: 10 - DNA, 804 bp, Homo sapiens
gaaggacgcaggcagcaaagagtagtcagtcccttcttggctctgctgacactcgagcccacattccatcacctgctcccaatcatgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgcctgaggggtccagaagcttcgaggcccagcgacctcagtgggcccagtggggaggagcaggagcctgagccttgggaacatgcgtgtgacctctacagctacctcttctatggactggttattgccaaacagccacactgtgggactcttcttaacttaaattttaatttatttatactatttagtttttataatttatttttgatttcacagtgtgtttgtgattgtttgctctgagagttccccctgtcccctccaccttccctcacagtgtgtctggtgacaaccgagtggctgtcatcggcctgtgtaggcagtcatggcaccaaagccaccagactgacaaatgtgtatcagatgcttttgttcagggctgtgatcggcctggggaaataataaagatgttcttttaaacggtaaaaaaaaaaaaaaaaaaaaaaaa

SEQ ID NO: 11 - DNA, 3136 bp, Homo sapiens
gcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 12 - DNA, 1909 bp, Homo sapiens
gaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaaaaaaaaaaaaaaaaaaaaaaaaaaaaaaaaaaaaaaaaaaaaaaaaaaa

(19) United States
(12) Patent Application Publication
(10) Pub. No.: US 2012/0192878 A1
(43) Pub. Date: Aug. 2, 2012

(54) METHODS OF DIAGNOSING AND MONITORING REJECTION MEDIATED BY ANTIBODIES

(75) Inventor: Mieko Toyoda, Fullerton, CA (US)

(73) Assignee: CEDARS-SINAI MEDICAL CENTER, Los Angeles, CA (US)

(21) Appl. No.: 13/501,434

(22) PCT Filed: Oct. 13, 2010
(86) PCT No.: PCT/US10/52559

§ 371 Date: Apr. 11, 2012

Related U.S. Application Data
(60) Provisional application No. 61/251,263, filed on Oct. 13, 2009.

(51) Int. Cl. C12Q 1/68; C40B 30/04; A61B 17/00; G01N 33/566

(52) U.S. Cl. 128/898; 435/6.11; 435/7.24; 435/7.1; 435/7.92; 506/9; 435/6.12

(57) ABSTRACT

An intracellular cytokine flow cytometry (CFC) assay was developed to measure CD3− (non-T) cell response to allo-Ags expressed on peripheral blood mononuclear cells (allo-CFC-PBMC) and/or endothelial cells (allo-CFC-EC) by detecting intracellular gamma-interferon (IFN_{Y}) production. The assay can be used to determine a likelihood of antibody mediated rejection in an individual. A method for using genetic screening to determine the likelihood of AMR is also disclosed.

### CROSS-REFERENCE TO RELATED APPLICATIONS

This application claims priority from U.S. Provisional Patent Application No. 61/251,263, filed on Oct. 13, 2009, which is incorporated herein by reference in its entirety.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. U01 AI46134 from the NIAID of the NIH.

### FIELD OF THE INVENTION

The invention generally relates to methods of monitoring and predicting antibody mediated rejection by means of cytokine flow cytometry (CFC) and genetic screening.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Certain antibodies in the blood of patients who receive an organ transplant pose a significant risk to the transplanted organ. These antibodies and the immune cells that produce them are directed against the donor's HLA antigens, protein components of the immune system. When present, these antibodies and cells can lead to immediate or delayed loss of a transplanted organ. These antibodies and cells exist in 30-40% of patients awaiting transplantation and may result from previous transplantations, transfusions and/or pregnancies.

Intravenous immunoglobulin (IVIG) was introduced to treat these patients by reducing these antibodies and cells, and many patients can now receive lifesaving transplantation. However, 20-30% of patients still develop rejection and many lose their organs. To know if IVIG-treated patients are ready to receive transplantation and to predict development of this type of rejection after transplant, there is a need in the art for novel methods of detecting the presence of immune cells that may cause organ transplant rejection together with antibodies, as well as other indicators.

### SUMMARY OF THE INVENTION

In one embodiment, the present invention provides a method of diagnosing susceptibility to a condition caused by any mechanism involving rejection mediated by an antibody (RMA), in an individual, including: obtaining a biological sample from the individual; assaying the sample to determine the presence or absence of an abnormal level of expression, relative to an individual who does not have ADCC in a kidney allograft or RMA, of one or more of the genes described in FIG. 17 and/or FIG. 37 herein; and diagnosing susceptibility to the condition based upon the presence of an abnormal level of expression, relative to an individual who does not have ADCC in a kidney allograft or RMA, of the one or more genes described in FIG. 17 and/or FIG. 37 herein. In certain embodiments susceptibility to RMA is determined prior to and/or after a subject receives an organ transplant.

In another embodiment, the present invention provides a method of diagnosing susceptibility to a condition caused by any mechanism involving rejection mediated by an antibody (RMA), in an individual, including: obtaining a biological sample from the individual; assaying the sample to determine the presence or absence of an elevated allo-antigen response; and diagnosing susceptibility to the condition based upon the presence of an elevated allo-antigen response. In certain embodiments, assaying the sample includes using cytokine flow cytometry to determine an allo-antigen response to peripheral blood mononuclear cells (PBMC) and/or endothelial cells (EC) and/or flow or luminex beads coated with natural or recombinant HLA antigens. In certain embodiments, the allo-antigen response is determined by the detection of IFNγ producing cells. In certain embodiments, the sample comprises Natural Killer (NK) cells. In certain embodiments, the sample comprises CD3− cells. In certain embodiments, the IFNγ producing cells are CD3− cells. In certain embodiments, the biological sample is blood. In certain embodiments, the biological sample includes: blood, sera, plasma, or combinations thereof. In certain embodiments, the biological sample including: biopsied kidney tissue. In certain embodiments, the individual is a female with a history of pregnancy. In certain embodiments, the PBMC and/or EC are derived from a prospective organ donor and/or one or more non-donors. In certain embodiments, the individual is a female with a history of pregnancy. In certain embodiments, the method also provides for determining the FCγRIIIa genotype of the individual, wherein if the individual has negative or low anti-allo reactivity and a FCγRIIIa-FF genotype then the susceptibility to the condition is a relatively low risk of developing the condition, and wherein if the individual is positive for one allo-CFC and has a genotype of FcγRIIIa-FF or VF then susceptibility to the condition is a relatively moderate risk of developing the condition, and wherein if the individual is positive for at least one allo-CFC and has a FcγRIIIa-VV genotype then the susceptibility to the condition is a relatively high risk of developing the condition.

In another embodiment, the present invention provides a method of transplanting an organ to an individual, including: diagnosing a lack of susceptibility to a condition caused by any mechanism involving rejection mediated by an antibody (RMA), according to the methods described herein, in the individual; and transplanting the organ to the individual. In certain embodiments, the organ is a kidney. In certain embodiments, the organ includes: a whole organ, a portion of an organ, skin, or other tissue or tissues suitable for transplant.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with an embodiment herein, IVIG-Rituximab-DES followed by transplantation and blood sample collection.

FIG. 2 depicts, in accordance with an embodiment herein, anti-allo reactivity in CD3-cells in response to various peripheral blood mononuclear cells (PBMCs) pre-desensitization (DES) in 55 highly sensitized (HS) patients and 14 normal individuals. HS patient blood were stimulated with PBMCs obtained from donor (ABOcom), 3rdN-ABOcom or 3rdN-ABOincom. 3rdN blood were stimulated with PBMCs obtained from HS patient (ABOcom), donor (ABOcom), or 3rdN-ABOincom.—and SEB represent negative (without stimulation) and positive controls (with super antigen), respectively. A horizontal dotted line represents anti-allo reactivity 5.0 and the ratio >5.0 represents high positive response. *: p<0.001, **: p<0.01, NS: not significant.

FIG. 3 depicts, in accordance with an embodiment herein, anti-allo reactivity in CD3-cells in response to various PBMCs pre-DES in 55 HS patients. All the anti-allo reactivity results in HS patients in FIG. 2 herein were plotted separately by gender, with or without transplant (Tx), and with or without AMR. *: p<0.05, NS: not significant.

FIG. 4 depicts, in accordance with an embodiment herein, anti-allo reactivity in CD3-cells in response to various allo-PBMCs in 8 normal females with a history of pregnancy, 8 females without a history of pregnancy and 5 normal males. Each individual blood was tested against PBMCs obtained from multiple individuals. The reactivity against each PBMC was tested multiple times and each data point in the figure was the average of the multiple results against each PBMC.

FIG. 5 depicts, in accordance with an embodiment herein, anti-allo reactivity in CD3-cells in response to various allo-PBMCs in 2 normal females w/PG. Blood was tested against PBMCs obtained from 15 individuals (10 PBMCs/female), and the reactivity against some of PBMCs was tested multiple times. Females with a history of pregnancy #1 and #2 correspond to those in FIG. 4 herein.

FIG. 6 depicts, in accordance with an embodiment herein, association between anti-allo reactivity in response to donor PBMC (anti-donor reactivity) and PRA-Class I (A), Class II (B) or donor-specific B cell-CMX (C) in 51 additional HS patients awaiting living donor transplantation before DES. A dotted line and c.c. represents a correlation line and correlation coefficient, respectively. MCS means mean channel shift.

FIG. 7 depicts, in accordance with an embodiment herein, IFNγ producing CD3− cells in response to allo-PBMCs in allo-CFC-PBMC. Lymphocytes first gated by forward/side scatter (A) were further plotted against CD3 and CD8 (B). CD3− cells (non-T cells including B and NK cells) were further plotted against IFNγ and IFNγ+ cell % in CD3− cells was calculated in a standard allo-CFC-PBMC (C). In allo-CFC-PBMC using additional fluorescence-conjugated antibodies, CD3− cells were plotted against IFNγ and CD19 or CD20 (CD19/CD20) (D).

FIG. 8 depicts, in accordance with an embodiment herein, IFNγ production in NK cell subsets in HS patient blood stimulated with allo-PBMCs (upper graphs) and alone without stimulation (lower graphs) in allo-CFC-PBMC. Since NK cells can be divided into several subsets by CD56 and CD16 expression, CD3− cells were plotted against CD16 and CD56 (left) and each NK subset (A, B or C) was plotted against IFNγ.

FIG. 9 depicts, in accordance with an embodiment herein, anti-allo reactivity with and without B cell depletion. B cells are depleted from whole blood using anti-CD19 coated dynabeads. Original whole blood and B cell depleted blood were submitted for lymphocyte subset analysis by flow cytometry (left) and allo-CFC-PBMC (right). NS mean not significant.

FIG. 10 depicts, in accordance with an embodiment herein, anti-allo reactivity pre- and post-B cell depletion by Rituximab in 25 HS patients. Blood samples were obtained pre- and post-DES (post-Ritux) and submitted for lymphocyte subset analysis by flow cytometry (A, C, D and E) and allo-CFC-PBMC (B). NS means not significant.

FIG. 11 depicts, in accordance with an embodiment herein, immune cell numbers pre-, post-DES and post-Tx in 11 HS patients treated with Campath 1H. Blood samples were obtained pre-, post-DES and post-Tx, and submitted for lymphocyte subset analysis by flow cytometry.

FIG. 12 depicts, in accordance with an embodiment herein, anti-allo reactivity in CD3-cells in modified allo-CFC-PBMC. 1) Plasma was separated from whole blood from HS patients with (+)CFC to particular allo-PBMC or 3rdN with (−)CFC to the allo-PBMC (cHS or cNC), 2) The allo-PBMC (stimulator) was pre-incubated with serum from the HS (sHS) and the NC (sNC), and 3) Allo-CFC-PBMC was performed using cHS and cNC, instead of using whole blood from HS or NC, against pre-treated allo-PBMC with sHS (PBMC w/sHS) or sNC (PBMC w/sNC).

FIG. 13 depicts, in accordance with an embodiment herein, Table 1 of anti-allo-reactivity to various PBMCs, anti-HLA antibody levels and specificity in 2 females with PG and HLA types in their family members and 3^{rd} party PBMCs.

FIG. 14 depicts, in accordance with an embodiment herein, Table 2 of anti-allo reactivity and DSA at various episodes in 37 living donor transplant patients.

FIG. 15 depicts, in accordance with an embodiment herein, Table 3 of allograft rejection and graft survival in AECA (+) and AECA (−) cardiac transplant recipients.

FIG. 16 depicts, in accordance with an embodiment herein, Table 4 of anti-allo reactivity in CD3− cells against allo-ECs as analyzed by a modified allo-CFC-EC.

FIG. 17 depicts, in accordance with an embodiment herein, a table of genes upregulated or downregulated in ADCC compared to the baseline. The data was generated as a result of a microarray performed for three (3) conditions: 1) BO (blood only, baseline), 2) MLR (mixed lymphocyte reaction: one type of allo-activation conditions), and 3) HS (highly sensitization, which means the ADCC—antibody-dependent cell cytotoxicity—condition). The 3 conditions were set up using blood obtained from 4 individuals. Allo-cfc assay assesses the degree of HLA sensitization and predicts AMR by measuring IFN levels by flow cytometry. The inventors found that IFN detected in the allo-cfc assay is due to ADCC that is different from MLR in mechanism: ADCC occurs through Fc gamma receptor bearing cells such as NK cells, while MLC occurs through T cells. The identification of specific genes expressed during (at) ADCC which may be used as markers for monitoring, allow prediction of AMR through a patient's urine instead of being limited to blood or biopsy. A total of 30,000 transcripts were in an array.

FIG. 18 depicts, in accordance with an embodiment herein, protocol for an anti-donor CFC experiment.

FIG. 19 depicts, in accordance with an embodiment herein: in A) Table 1 a summary of anti-donor-CFC reactivity and DSA at various clinical episodes post-Tx in 36 HS patients, and B) Table 2 shows anti-donor-CFC reactivity and DSA at each episode of AMR, CMR or ATN in 13 HS patients in this study. Anti-donor-CFC was (+) in most HS patients at AMR with DSA (++), all HS w/ATN and some w/other clinical problems, often with DSA present. Most stable patients showed anti-donor-CFC (−) and DSA (−).

FIG. 20 depicts, in accordance with an embodiment herein, A) 2 female and B) 5 male HS patients who developed AMR. One of the two female patients showed negative result, but anti-donor-CFC reactivity increased prior to and/or at AMR (arrow). Elevated anti-donor-CFC reactivity was observed prior to and/or at AMR in all the 5 male patients. The dotted line represents high (+) anti-donor-CFC cut-off level in females and males. The red vertical line separate pre-Tx and post-Tx.

FIG. 21 depicts, in accordance with an embodiment herein, a typical CFC assay result: upper and lower graphs represent flow cytometry analysis results in blood without (negative control) and with allo-PBMCs, respectively. Lymphocytes first gated by forward and side scatter (FSC/SSC) (A) were further plotted against CD3 and CD8 (B). CD3−, CD3+/CD8+ and CD3+/CD8−cells were then plotted against IFNγ (C, D) and IFNγ+cell % in each cell population was calculated. Results of the CFC assay were expressed as the ratio of IFNγ+cell % in blood with vs. without allo-PBMCs. Lymphocyte % in total cells acquired, CD3−, CD3+/CD8+ and CD3+/CD8−cell % in the lymphocyte gate in 182 CFC assays tested in this study were 21.4±5.5%, 34.5±8.4%, 25.4±6.5% and 39.8±6.8%, respectively, in blood without PBMC stimulation and 27.7±6.7%, 29.9±6.9%, 25.3±5.0% and 44.7±5.6% in blood with PBMC stimulation.

FIG. 22 depicts, in accordance with an embodiment herein, anti-allo reactivity in CD3− (A), CD3+/CD8+ (B) and CD3+/CD8− cells (C) in response to various allo-PBMCs in 8 normal females with pPG, 8 females without pPG and 5 normal males. Each individual whole blood was tested for anti-allo reactivity against PBMCs obtained from multiple individuals. The reactivity against each PBMC was tested multiple times (2.2±2.2 times/PBMC, range 1-12), and each data point in the figure was the average of the multiple results against each PBMC. In 182 CFC assays tested in this study, median IFNγ+ cell % in CD3− cells in blood without and with allo-PBMC were 0.6% (range 0.1-3.3%) and 6.5% (0.1-19.2%) in 4 females with pPG with elevated anti-allo reactivity (#1-4), 0.5% (range 0.3-4.9%) and 0.4% (0.2-1.5%) in 4 females with pPG without elevated anti-allo reactivity (#5-8), 1.1% (range 0.01-3.7%) and 1.1% (0.01-3.4%) in 8 females without pPG, and 0.8% (range 0.1-8.6%) and 0.5% (0.1-4.0%) in 5 males, respectively. IFNγ+ cell % in CD3+/CD8+ cells in all blood samples tested without and with allo-PBMC ranged from 0.01% to 2.3% and from 0.03% to 4.2%, and those in CD3+/CD8− cells ranged from 0.01% to 1.8% and from 0.01% to 2.8%, respectively.

FIG. 23 depicts, in accordance with an embodiment herein, correlation of anti-allo reactivity in CD3−cells vs. CD3+/CD8+ (A) or CD3+/CD8−cells (B). Of 182 CFC assays, 158 (56, 54 and 48 in females with pPG, without pPG and males, respectively) were tested against PBMCs obtained from single donor and included in this analysis.

FIG. 24 depicts, in accordance with an embodiment herein, anti-allo reactivity in CD3−cells in response to various allo-PBMCs in 2 normal females with pPG. Whole blood was tested for anti-allo reactivity against PBMCs obtained from 15 individuals (10 PBMCs/female), and the reactivity against some of PBMCs was tested multiple times. Females with pPGs #1 and #2 correspond to those in FIG. 21.

FIG. 25 depicts, in accordance with an embodiment herein, anti-allo reactivity in CD3−cells in response to the same PBMC over years in 2 normal females with pPG. Anti-allo reactivities tested multiple times in females with pPG #1 (closed symbols with solid line) and #2 (open symbols with dotted line) in FIG. 2 were plotted by date tested. Various symbols describe anti-allo reactivity against different PBMCs (PBMC #1—circle, #2—triangle, #3—large diamond, #4—small diamond and #6—square in female with pPG #1; PBMC #3—diamond, #4—triangle and #11—circle in female with pPG #2).

FIG. 26 depicts, in accordance with an embodiment herein, the association between anti-allo reactivity in CD3−cells and anti-HLA class I or class II antibody levels as analyzed by ELISA in 7 females with pPG, 6 females without pPG and 5 males. Each individual whole blood was tested for anti-allo reactivity against PBMCs obtained from multiple individuals and multiple times (2.2±2.2 times/PBMC, range 1-12). Anti-HLA antibody level was measured at each time point when anti-allo reactivity was tested. Each data point in the figure was the average of all the results in each individual. A dotted line represents the high normal limit of anti-HLA antibody class I (292 units) and class II (209 units).

FIG. 27 depicts, in accordance with an embodiment herein anti-allo reactivity in CD3− cells in response to allo-PBMCs and anti-HLA antibody levels in 2 normal females with pPG over years. Females with pPGs #1 and #2 correspond to those in FIG. 21. Short dotted lines on the right y-axis represent the high normal limit of anti-HLA antibody class 1 (302 units) and class II (214 units). Arrows represent time points for anti-HLA antibody testing. PRA: panel reactive antibodies as analyzed by Luminex assay, DSA: HLA antibodies specific to antigenic pHLA-Ags shown in FIG. 13.

FIG. 28 depicts, in accordance with an embodiment herein, a diagram of an Allo-CFC assay.

FIG. 29 depicts, in accordance with an embodiment herein, A) an experiment and B) the results to determine if NK cell activation in the CFC is ADCC. Plasma (p) was separated from whole blood of 5 HS with CFC(+) (HSp) or 5 normal controls with CFC(−) (NCp). Blood cells from HS(HSc) or NC(NCc) (responders) were incubated with irradiated peripheral blood mononuclear cells (PBMCs) pre-treated with HSp or NCp (stimulators), and anti-allo reactivity was measured. Both HSc and NCc showed high(+) CFC against PBMCs pretreated with HSp, but not NCp (HSc: 6.1±2.3 vs. 1.4±0.9, p<0.01; NCc: 7.2±4.1 vs. 1.2±0.4, p<0.03). These results suggest that NK cell activation observed in the CFC is primarily via ADCC.

FIG. 30 depicts, in accordance with an embodiment herein, immune cell number in highly sensitized patients transplanted after desensitization with IVIG and rituximab. NK cell number were 0.62±0.33 or 0.45±0.18 in patients treated with Campath 1H or Zenapax, respectively, even in the 1st 3 months post-Tx when CD19+ (0.12±0.21) (pre-desensitization 1.0), CD4+ (0.07±0.15) and CD8+ (0.13±0.20) cells were nearly undetectable in HS w/Camp. While not wishing to be bound by any particular theory, these results suggest a possible role of NK cells in AMR in HS transplant recipients, There was no difference in NK cell number detected between patients w/campath 1H vs. zenapax induction, w/vs. w/o AMR, w/vs. w/o ATN or w/vs. w/o CMR (FIG. 31).

FIG. 31 depicts, in accordance with an embodiment herein, post-transplant immune cell numbers in highly HLA-sensitized living-donor patients transplanted after desensitization.

FIG. 32 depicts, in accordance with an embodiment herein, anti-donor-CFC reactivity and DSA at each episode of AMR, CMR or ATN in 13 highly HLA-sensitized (HS) patients transplanted after desensitization with IVIG and rituximab. Of 7 HS patients with AMR, 6 showed anti-donor-CFC(+) at or right before AMR with DSA (++) in most patients. 5 were diagnosed with ATN with C4d(−), and all had CFC(+) at biopsy with 2/5 DSA(++). 1/2 HS patients with CMR was CFC(+) with DSA(+). DSA levels at these episodes varied (<105 or >105 SFI). While not wishing to be bound by any particular theory, these results suggest that NK cells activated through ADCC mechanisms may contribute to AMR, ATN and CMR in HS transplant recipients.

FIG. 33 depicts, in accordance with an embodiment herein, a model for studying gene expression for antibody-dependent cell cytotoxicity (ADCC) in natural killer (NK) cells.

FIG. 34 depicts, in accordance with an embodiment herein, a table of kidney biopsy samples for the study.

FIG. 35 depicts, in accordance with an embodiment herein, ADCC-specific (a) and MLR-specific (b) gene expression patterns.

FIG. 36 depicts, in accordance with an embodiment herein, gene expression in kidney biopsies for IFNG, CCL3 and CCL4.

FIG. 37 depicts, in accordance with an embodiment herein, a total of 12 ADCC-specific genes that were identified in the study with >1.5-fold difference (p<0.05).

FIG. 38 depicts, in accordance with an embodiment herein, an additional 46 MLR-specific genes identified with >1.5-fold difference (p<0.05).

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

As used herein, “CMR” means cell mediated rejection.

As used herein, “DSA” means donor specific antibody.

As used herein, “EC” means endothelial cells.

As used herein, “NS” means not significant.

As used herein, “MCS” means mean channel shift.

As used herein, “PG” means history of pregnancy.

As used herein, “AMR” means antibody mediated rejection.

As used herein, “Tx” means transplant.

As used herein, “HS” means highly sensitized.

As used herein, “DES” means desensitization.

As used herein, “CFC” means cytokine flow cytometry.

As used herein, “ADCC” means antibody dependent cell cytotoxicity.

As used herein, “NK” means natural killer cell.

As used herein, “Ags” means antigens.

As used herein, “PBMC” means peripheral blood mononuclear cell.

As used herein, “3^{rd}N” means 3^{rd} party normal individuals.

As used herein, “PRA” means panel reactive antibodies.

As used herein, “antibody mediated rejection” means any rejection caused by any mechanism(s) involving antibodies.

As used herein, the term “biological sample” means any biological material from which nucleic acid molecules or protein can be prepared. As non-limiting examples, the term material encompasses whole blood, plasma, sera, saliva, cheek swab, or other bodily fluid or tissue that contains nucleic acid or protein.

As used herein, the term “abnormal expression” of genes refers to levels of expression, such as quantifiable levels of mRNA transcripts for example, that significantly differ from levels one of skill in the art would expect to find in a subject with normal expression, such as for example, a subject who does not have ADCC in a kidney allograft and is not exhibiting AMR.

As apparent to one of skill in the art, numerous versions exist for genetic loci described herein. Examples of genetic loci of interferon gamma, chemokine (C motif) ligand 2, chemokine (C-C motif) ligand 4, early growth response 2 (Krox-20 homolog, Drosophila), nuclear receptor subfamily 4, group A, member 3, chemokine (C-C motif) ligand 4-like 1, CD160 molecule, chemokine (C-C motif) ligand 3, cytotoxic and regulatory T cell molecule, chemokine (C-C motif) ligand 3-like 1, early growth response 1, and Fas ligand (TNF superfamily, member 6), as described herein as Seq ID Nos. 1, 2, 3, 4, 5, 6, 7, 8, 9, 10, 11 and 12, respectively, although the loci are in no way limited to these specific sequences.

As disclosed herein, high rates of antibody mediated rejection (AMR) remain an obstacle in highly sensitized (HS) patients receiving desensitization (DES) followed by transplant (Tx). Current screening methods for AMR consist primarily of antibody assays, but tools measuring cell-mediated immunity to allo-antigens (Ags) are lacking. The inventors developed an intracellular cytokine flow cytometry (CFC) assay to measure CD3− (non-T) cell response to allo-Ags expressed on peripheral blood mononuclear cells (allo-CFC-PBMC) by detecting intracellular gamma-interferon (IFNγ) production. The inventors found the following: 1) Allo-Ag-specific CD3− cells were elevated in many HS patients and 2) HS patients with high(+) allo-CFC-PBMC pre-Tx were at high risk for AMR, and elevated allo-CFC-PBMC was observed prior to AMR post-Tx in some patients. These findings demonstrate the utility of the allo-CFC-PBMC for predicting AMR. Additionally, the inventors found allo-Ag-specific CD3− cells detected in the allo-CFC-PBMC were primarily NK cells. This NK cell response is primarily due to antibody dependent cell cytotoxicity (ADCC), showing NK cell involvement in development of AMR.

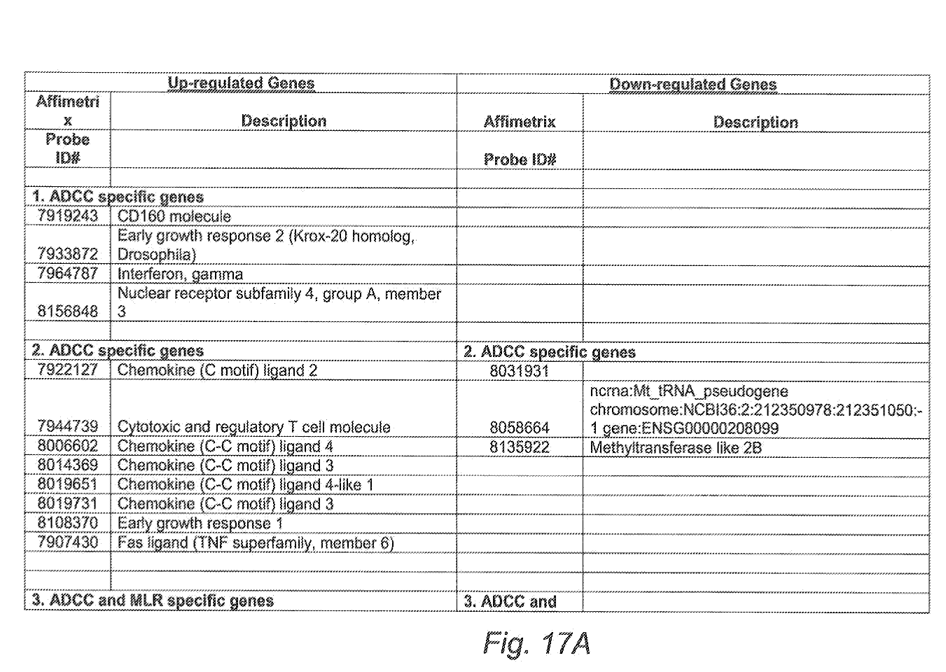

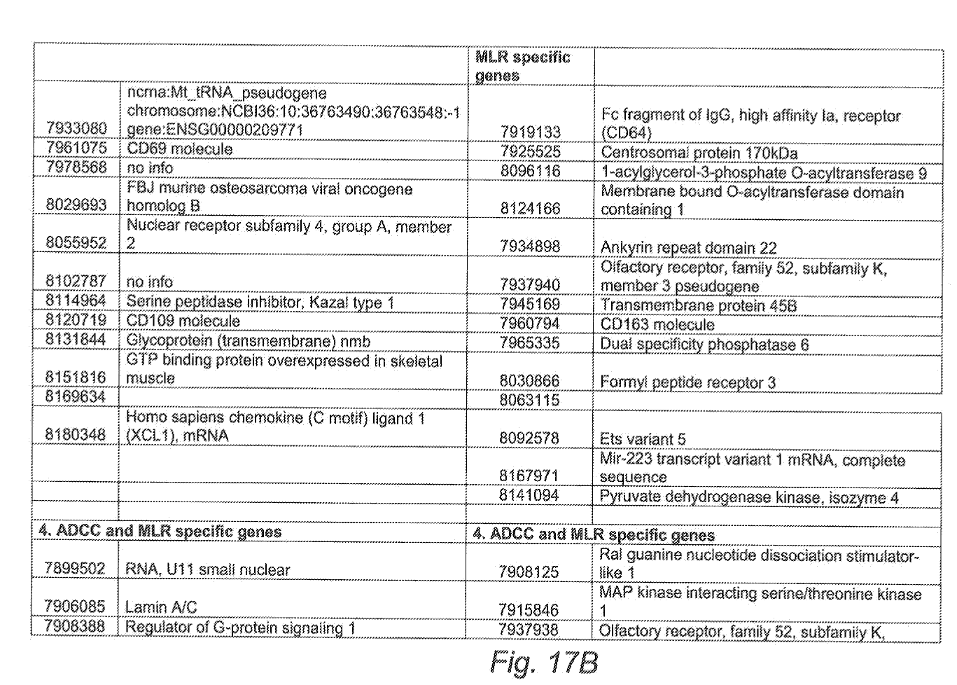

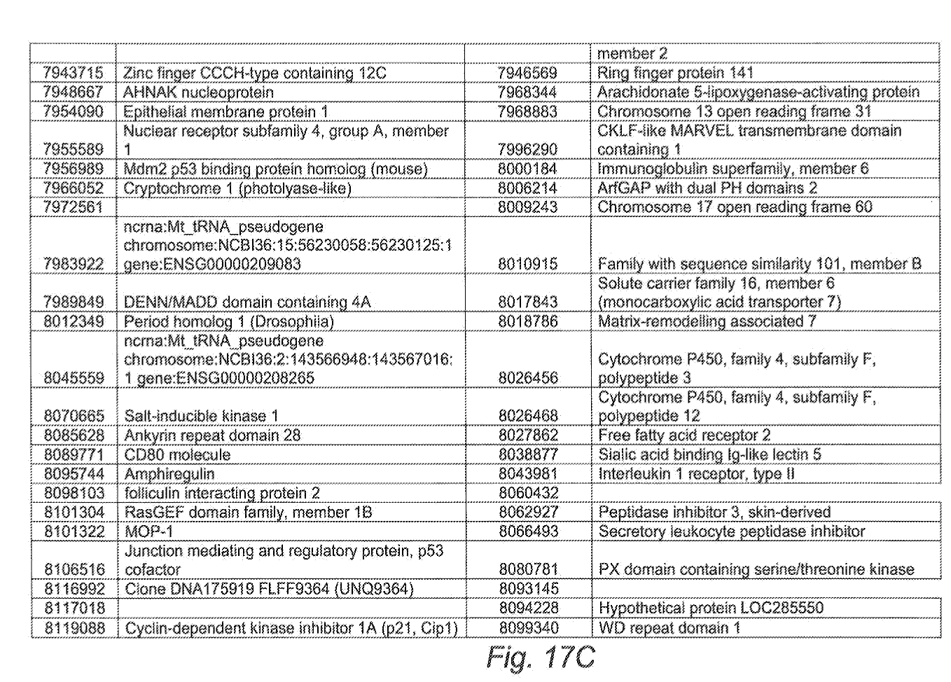

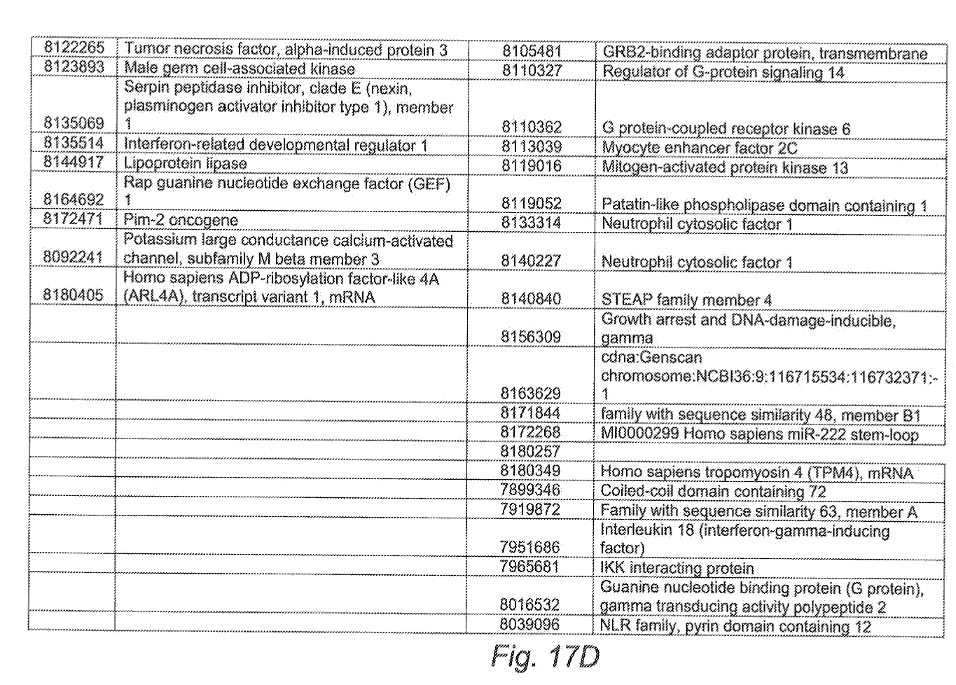

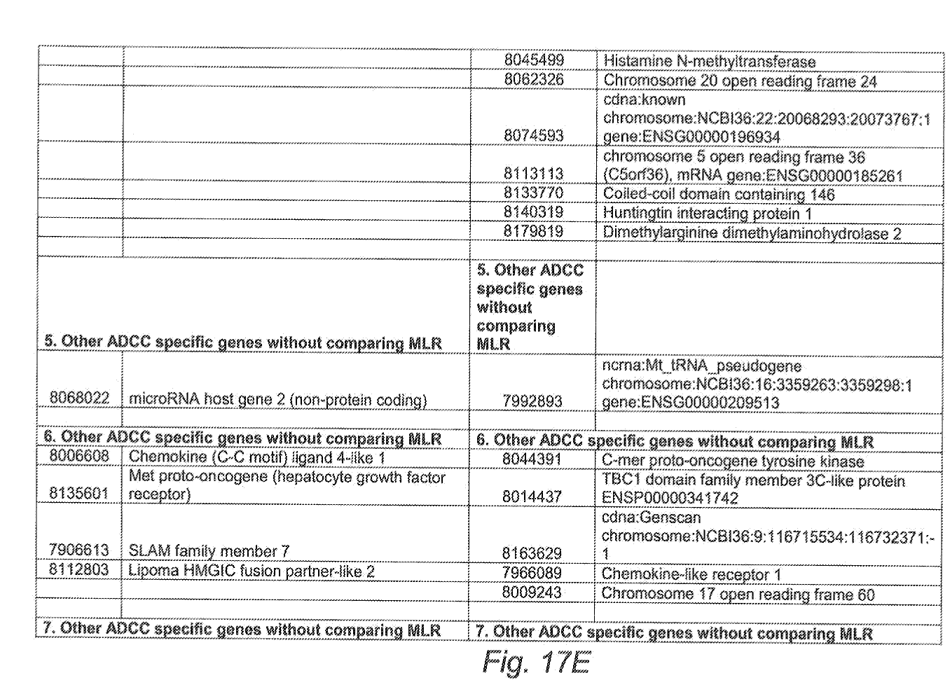

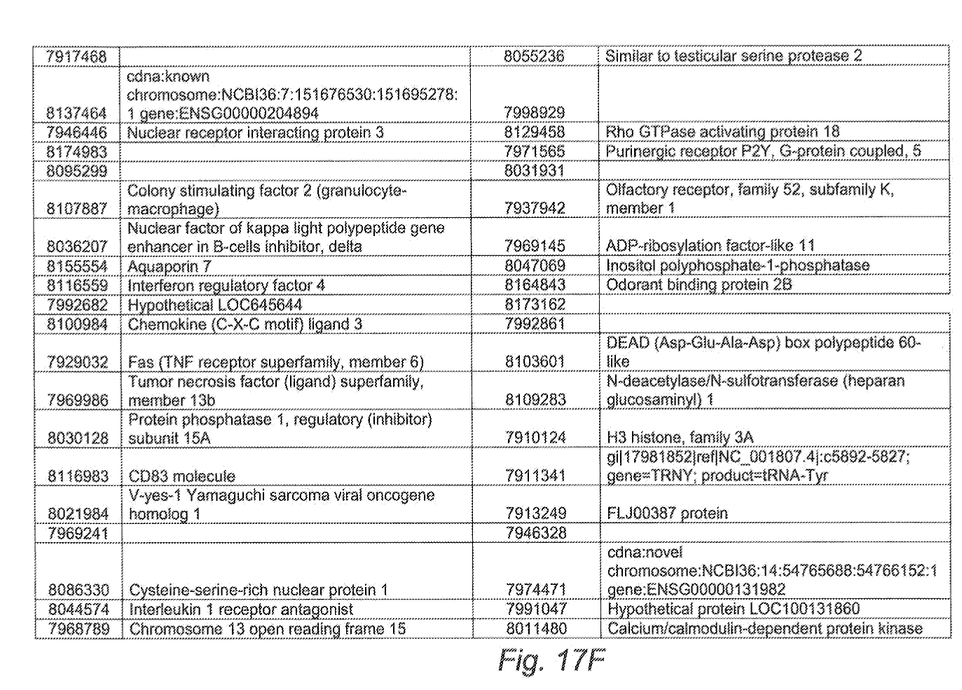

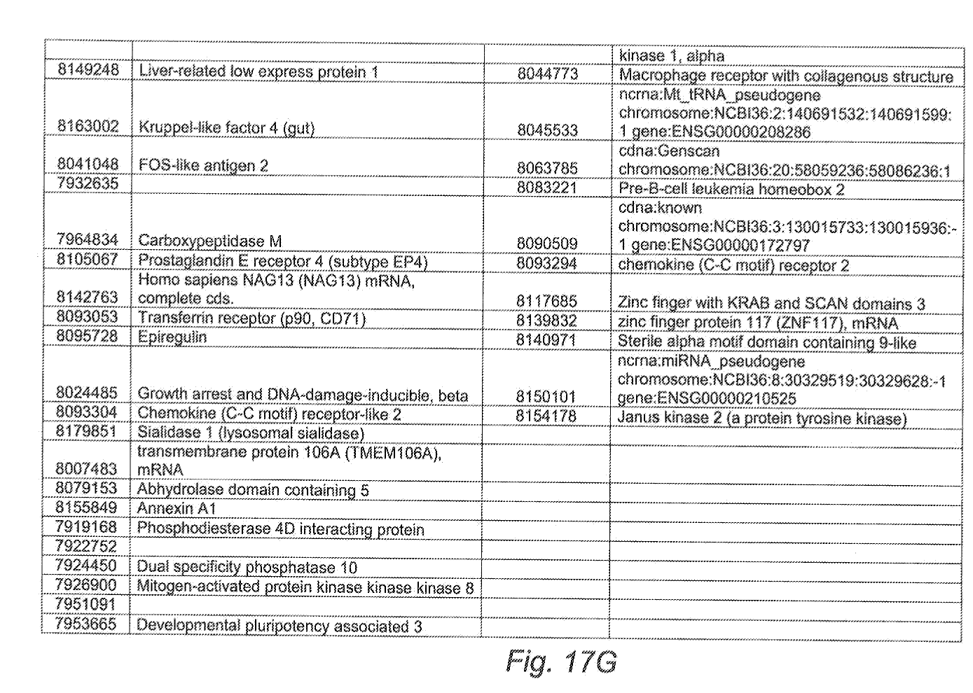

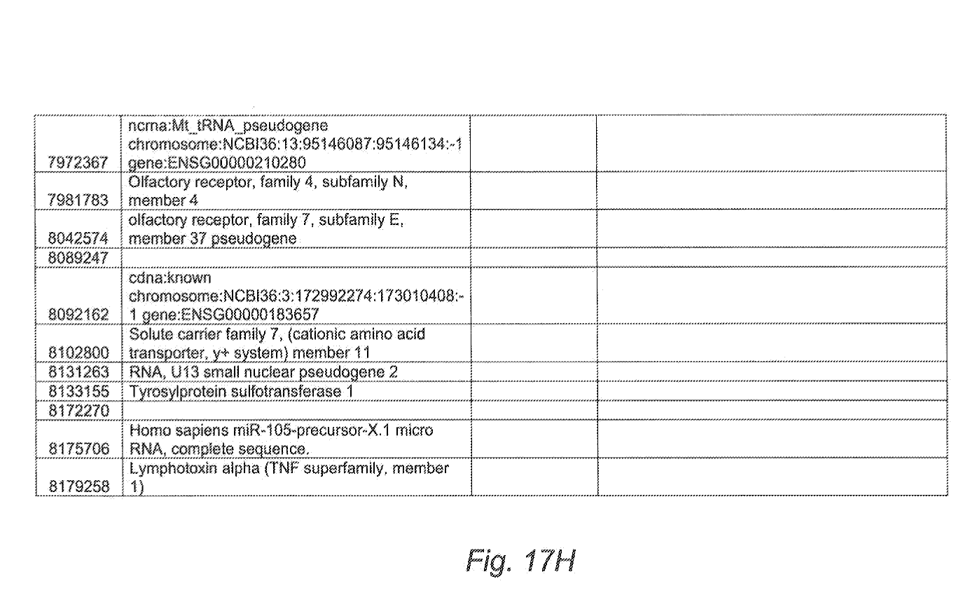

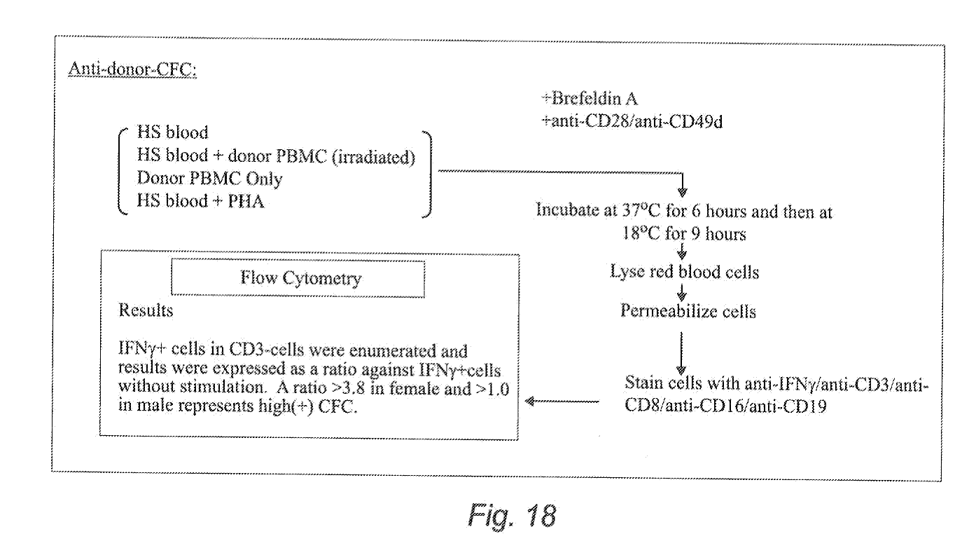

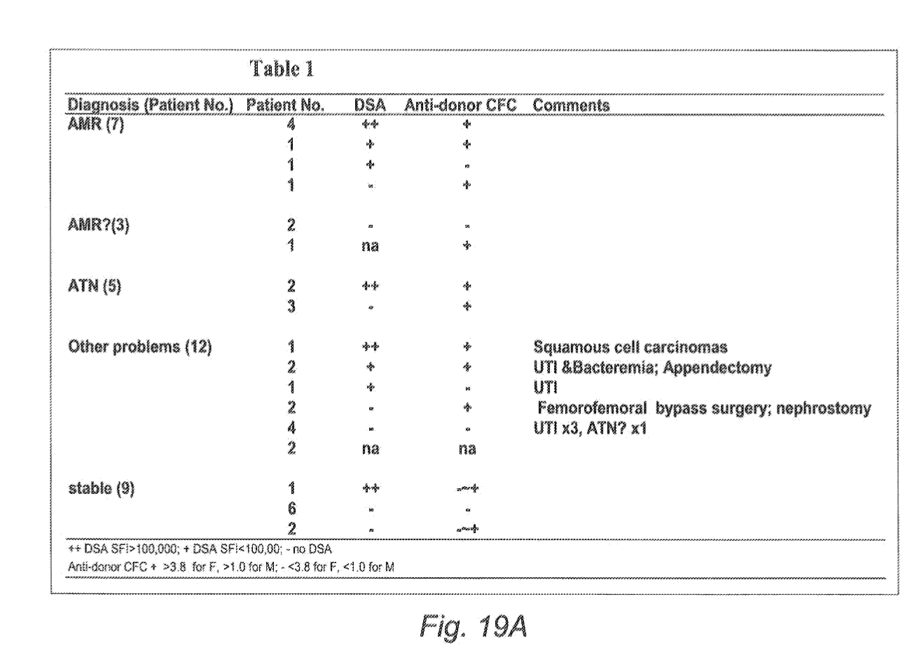

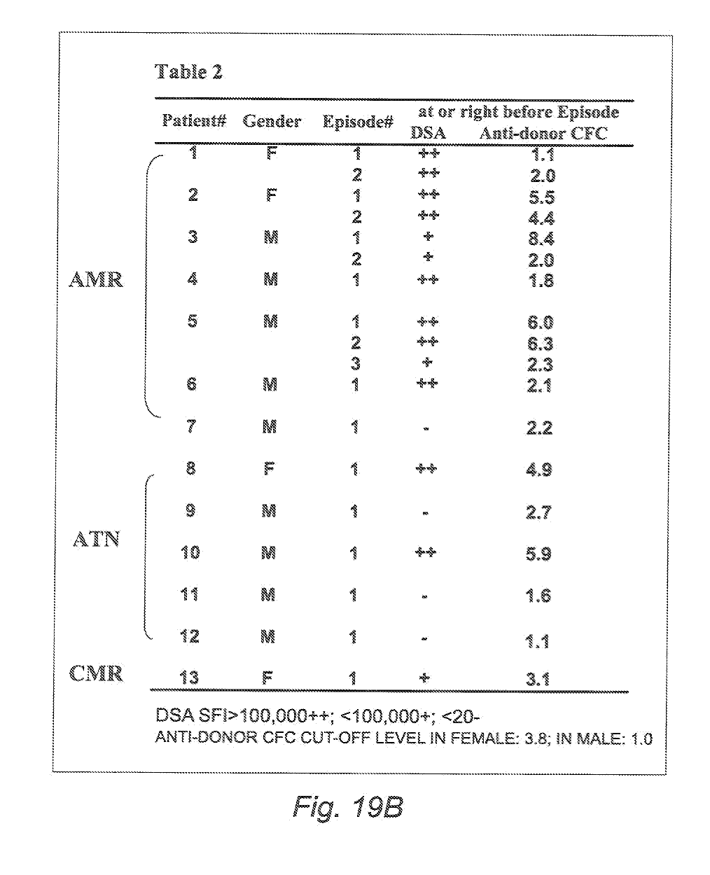

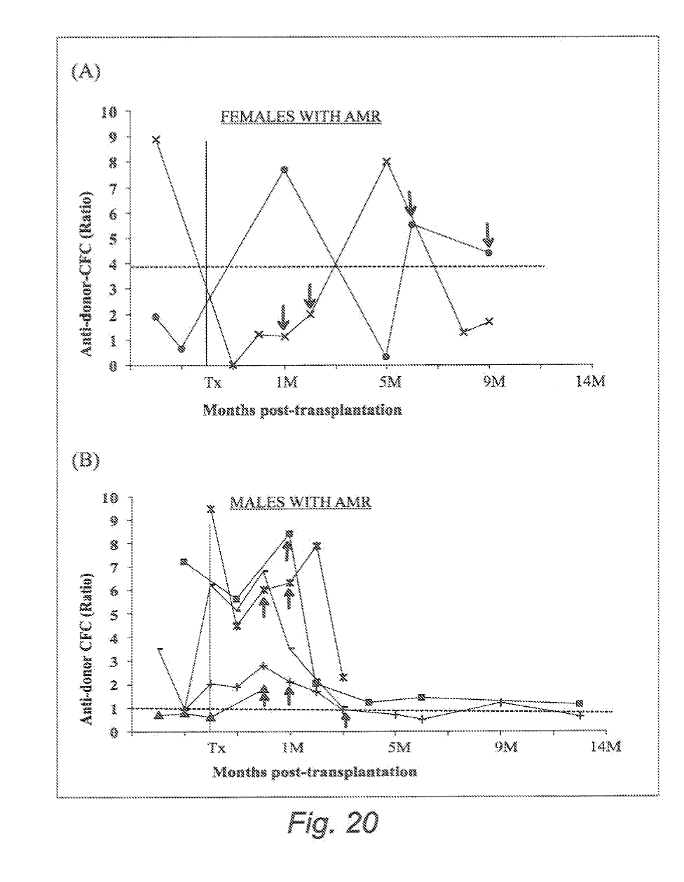

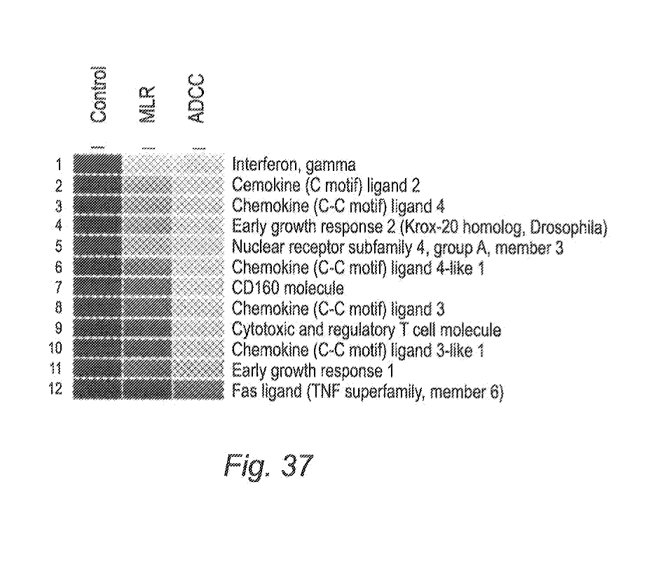

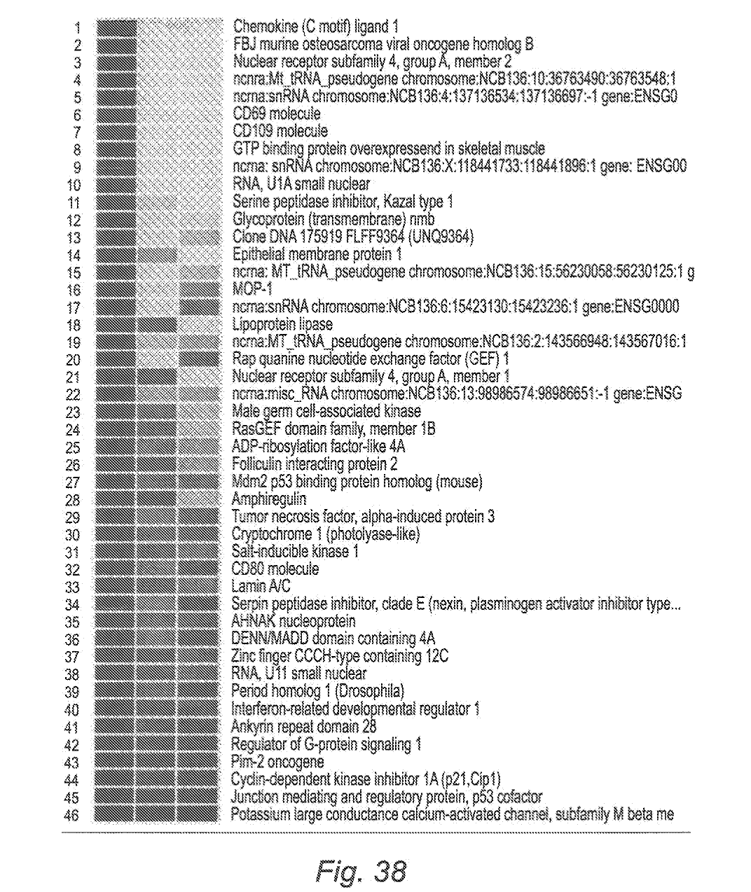

As further disclosed herein, the inventors performed microarray analysis in the allo-CFC-PBMC setting to identify specific genes expressed in ADCC, and found about 20 ADCC-specific genes including IFNγ. From their findings, mRNA expression of these genes is elevated in urine of patients who have ADCC in the kidney allograft, resulting in development of AMR. mRNA expression of these genes was detected in kidney biopsies obtained from HS patients. Biopsies not only from patients with AMR but also those with other kidney injuries such as CMR and ATN (acute tubular necrosis) showed positive for expression of these genes. As described in FIG. 17 and/or FIG. 37 herein, various genes are upregulated or downregulated in ADCC compared to the baseline, thus having abnormal expression levels relative to an individual who does not have ADCC in the kidney allograft or AMR. While not wishing to be bound by any particular theory, evidence suggests it is possible to detect the expression of these genes earlier and more consistently in urine compared to blood since ADCC occurs primarily in the kidney allograft and not systemically. Alternatively, detection using samples from kidney biopsies may be useful in accurately diagnosing kidney injuries caused by ADCC. This will allow an accurate and earlier prediction of AMR.

In one embodiment, the present invention provides a method of diagnosing and/or predicting susceptibility to antibody mediated rejection (AMR) in an individual by determining the presence or absence of an abnormal expression, relative to an individual who does not have ADCC in a kidney allograft or AMR, of the one or more genes described in FIG. 17 and/or FIG. 37 herein, where the presence of an abnormal expression relative to an individual who does not have ADCC in a kidney allograft or AMR of the one or more genes described in FIG. 17 and/or FIG. 37 herein is indicative of susceptibility to AMR. In another embodiment, abnormal expression levels of the one or more genes described in FIG. 17 and/or FIG. 37 may be determined and/or monitored by examining the urine or other biological materials of the individual. In certain embodiments the biological materials include: blood, sera, plasma, saliva, urine or tissue derived from a biopsy or otherwise. In certain embodiments, the expression levels of the one or more genes may be determined and/or monitored by amplifying transcripts. In another embodiment, the transcripts may be amplified by RT-PCR. In certain embodiments, the individual has ADCC in a received kidney allograft. In certain embodiments, the individual is tested for susceptibility to AMR prior to organ transplantation. In certain embodiments the individual is tested during desensitization. In another embodiment the individual is tested after organ transplantation.

In another embodiment, relative to levels found in a an individual who does not have ADCC in a kidney allograft or AMR, the upregulation of expression of one or more genes selected from the group comprising CD160 molecule, early growth response 2, interferon gamma and nuclear receptor subfamily 4, the upregulation of expression of one or more genes selected from the group comprising mt tRNA pseudogene, CD69 molecule, FBJ murine osteosarcoma viral oncogene homolog B, nuclear receptor subfamily 4, serine peptidase inhibitor, CD109 molecule, glycoprotein (transmembrane), GTP binding protein overexpressed in skeletal muscle, homo sapiens chemokind (C motif) ligand 1, and/or the downregulation of expression of one or more genes selected from the group comprising Fc fragment of IgG, centrosomal protein, 1-acylglycerol-3-phosphate O-acyltransferase 9, membrane bound O-acyltransferase domain containing 1, ankyrin repeat domain 22, Olfactory receptor family 52 subfamily K member 3 pseudogene, transmembrane protein 45B, CD163 molecule, CD163 molecule, dual specificity phosphatase 6, formyl peptide receptor 3, matrix metallopeptidase 9, ets variant 5, mir-223 transcript variant 1 mRNA, pyruvate dehydrogenase kinase, is indicative of susceptibility to AMR. In another embodiment, relative to an individual who does not have ADCC in a kidney allograft or AMR, the upregulation of expression levels of microRNA host gene 2 and/or downregulation of expression levels of mt tRNA pseudogene chromosome, is indicative of susceptibility to AMR. In another embodiment the genes that when up-regulated are associated with AMR include all genes listed under “up-regulated genes” in FIG. 17. In another embodiment down-regulated genes associated with AMR include those listed under “down-regulated genes” in FIG. 17. In another embodiment, abnormal expression of the genes in FIG. 37 is associated with ADCC.

As further disclosed herein, although the allo-CFC-PBMC predicts AMR, 10-20% of patients showed discordant results: patients with low allo-CFC-PBMC developed AMR, and vice versa. While not wishing to be bound by one particular theory, these results suggest that other factors should be considered in adjunct to allo-CFC-PBMC to better predict AMR. Thus, the inventors developed a more comprehensive protocol to predict AMR by the following: 1) instituting allo-CFC using endothelial cells (ECs) as antigenic targets (allo-CFC-EC) since ECs express Ags different from PBMCs; and 2) measuring CD16 (FcγRIIIa) polymorphism since the FcγRIIIa-158V genotype is known to have higher ADCC activity than the FcγRIIIa-158F genotype. Results may then be compared with allo-CFC-PBMC, AMR episodes, graft survival and other clinical data. Patients with negative allo-CFCs and FcγRIIIa-FF or VF genotype will have AMR-free Tx, those positive for one allo-CFC and FcγRIIIa-VV genotype are likely to develop AMR, and those positive for one allo-CFC and FcγRIIIa-FF or VF genotype are at moderate risk for AMR. This can be applied both pre- and post-Tx, thus providing an important tool for monitoring patients.

In one embodiment, the present invention provides a method of diagnosing and/or predicting susceptibility for antibody mediated rejection (AMR) in an individual by monitoring allo-antigen responses while the individual undergoes desensitization, where an elevated allo-antigen response is indicative of susceptibility to AMR. In another embodiment, the desensitization is performed by using the IVIG procedure. In another embodiment, the elevated allo-antigen response comprises an elevated response of allo-antigen-specific CD3− cells. In another embodiment, the elevated allo-antigen response comprises an elevated response of NK cells. In another embodiment, susceptibility to AMR is determined by measuring CD16 (FCγRIIIa) polymorphisms. In another embodiment, the allo-antigen response is measured by cytokine flow cytometry. In another embodiment, the cytokine flow cytometry detects IFNγ produced in response to allo-antigen stimulation and quantifies the frequency of allo-antigen-specific responses. In another embodiment, the allo-antigen responses are measured by allo-CFC-PBMC and/or allo-CFC-EC. In certain embodiments the PBMC or EC samples are derived from the prospective donor. In certain embodiments the PBMC or EC samples are derived from non-donors. In still other embodiments PBMC or EC samples of various individuals are mixed and the mixed samples are used to test for anti-allo-antigen response. In certain embodiments, flow or luminex beads coated with natural or recombinant HLA antigens are used to test for an anti-allo antigen response. In certain embodiments the allo-antigen responses are evaluated in conjunction with the FCγRIIIa to determine the likelihood of AMR. In one embodiment, the individual tested for the likelihood of AMR rejection is a female with a history of pregnancy. In certain embodiments of the invention, the individual tested is a child. In certain embodiments of the invention the individual tested has a history of at least one blood transfusion.

In another embodiment, the present invention provides a method of transplanting an organ to an individual by diagnosing the individual as having a lack of susceptibility to AMR based on any test or combination of tests to determine susceptibility described herein and then transplanting the organ to the individual. In certain embodiments, the organ includes: a whole organ, a portion of an organ, skin, or other tissue or tissues suitable for transplant.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### General Methods

50 HS patients undergoing desensitization and receiving a kidney allograft are included. Measurements are taken of the following: i) IFNγ+ cell % in NK cells and other cell populations reactive with allo-PBMCs obtained from donor and 3^{rd} party normal individuals (3^{rd}N), and with allo-ECs obtained from 3^{rd}N by CFC in blood obtained pre- and post-DES, with frequent monitoring post-Tx, and ii) FcγRIIIa genotype by TaqMan SNP genotyping assay using DNA extracted from blood obtained pre-Tx. The follow-up period is 6 months post-treatment for all and 1 year for ⅔ of patients. The total duration is 2 years, with results compared with other clinical data.

### Example 2

### Methods—Patient Population

HS adult and pediatric patients who receive the DES protocol followed by a kidney allograft from a living ABO compatible or incompatible donor are included. Only living donor Tx are included since the time between DES and Tx fixed at approximately 1 month post-DES, while that in deceased-donor Tx is uncertain. At least 160 kidney Txs per year are performed (240/1.5 years). Of these, 40% are highly sensitized or scheduled to receive a kidney (100 patients/1.5 years). Of these, 50% receive a living-donor Tx (50 patients/1.5 years).

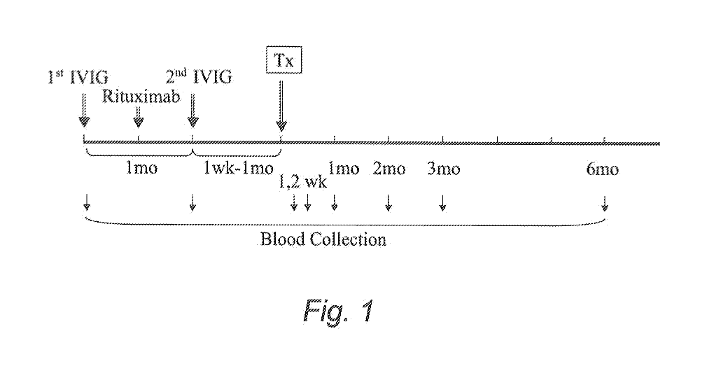

The DES protocol consists of 2 doses of IVIG (2 g/kg) one month apart with 1 dose of rituximab (1 g/dose) in between. The IVIG product used in this study is Gamunex-10% (Talecris Biotherapeutics). The flow chart of this study is shown in FIG. 1 herein.

Flow-CMX against donor is tested pre- and post-DES. When a negative or acceptable CMX (negative CDC-CMX and flow-CMX <250 mean channel shifts) is achieved, the patient is scheduled for Tx, usually within 1 week to 1 month after the 2^{nd} IVIG dose. Using this DES protocol and criteria, more than 90% of patients are able to receive Tx.

Immune Suppression is accomplished according to the following: Adult: [Induction] Campath 1H for high risk patients with historically high CMX+ or ABO incompatible Tx, and anti-IL-2 receptor for low risk patients with IV methyl prednisone. [Maintenance] FK506, MMF and prednisone. [For African American] 50% higher dose of FK506. Children: FK506, MMF and prednisone with induction therapy with anti-IL-2 receptor.

Anti-Rejection Treatment is accomplished according to the following protocol: one dose of IVIG (2 g/kg), rituximab (375 mg/m²) and/or plasmapheresis are applied for AMR, and IV pulse methyl prednisone for 3 days for CMR. If steroid resistant, OKT3 or Thymoglobulin for 7-10 days are applied.

### Example 3

### Methods—Sample Collection

15 ml of heparinized-blood are obtained from patients and submitted for allo-CFC-PBMC and allo-CFC-EC on the day of blood draw. Blood samples are obtained from each patient at 8 time points (pre-1^{st} IVIG, pre-2^{nd} IVIG, 1 and 2 weeks, 1, 2, 3 and 6 months post-Tx) (FIG. 1). 5 ml of EDTA-anti-coagulated-blood are obtained once from each patient and submitted for DNA extraction followed by FcγRIIIa genotype analysis.

Heparinised-whole blood is submitted for both allo-CFCs and the remaining blood is centrifuged to obtain plasma for anti-HLA antibody ELISA analysis. Leukocyte pellets are first prepared from the EDTA-anti-coagulated-blood by lysing red blood cells with ammonium carbonate/chloride as previously described and stored at −80° C. for batched DNA extraction and TaqMan SNP genotyping analysis.

### Example 4

### Methods—Measurement of Anti-Allo Reactivity by Allo-CFCs

The CFC detects intracellular IFNγ produced in response to Ag stimulation and quantifies the frequency of Ag-specific cell responses. The allo-CFCs are performed using whole blood as previously described with some modifications. PBMCs obtained from donor or 3^{rd}N, or ECs from 3^{rd}N are used as allo-Ags. Whole blood is incubated with irradiated allo-PBMCs in microcentrifuge tube or fixed/cryo-preserved ECs on 96-well plate, CD28/49d and Brefeldin A. Red cells are then lysed, leukocytes are fixed followed by permeabilization, and intracellular IFNγ and surface markers stained using an anti-IFNγ/anti-CD3/anti-CD8/anti-CD56/anti-CD16 antibody cocktail. The IFNγ+ cell % in CD3−, NK subset (CD56+, CD56+/CD16+, CD56+/CD16−, CD56−/CD16+), CD4+ and CD8+ cell populations is determined. In a previous allo-CFC, IFNγ+ cell % in CD3− cells was measured. The inventors now know that IFNγ+/CD3− cells detected in allo-CFC are primarily CD56+/CD16− NK cells originating from CD56+/CD16+ NK cells. Thus, measurements are made of IFNγ+ cell % in various NK cell subsets as described herein.

PBMCs are obtained from donor and 3^{rd}N are used as Ags for allo-CFC-PBMC. While not wishing to be bound by any one particular theory, although using donor PBMCs is preferred when measuring immune cell reactivity in a Tx setting, measuring the global immune cell reactivity against a mixture of 3^{rd}N PBMCs is also informative. The former is similar in concept to donor CMX, the latter to PRA. PBMCs are isolated from the donor and 5-10 3^{rd}N by density-gradient centrifugation using Ficoll-Hypaque. Donor PBMCs are aliquoted and frozen. 3^{rd} N PBMCs are individually isolated from blood, mixed, and then frozen.

HUVECs obtained from 3^{rd}N are used as Ags for allo-CFC-EC. Fixed/cryo-preserved ECs on 96-well plate used for AECA ELISA established by the inventors are used for this study. Briefly, HUVEC mixture isolated from at least 5 3^{rd}N obtained from Lonza (Basel, Switzerland) are cultured in EC media and passaged serially on plastic flasks. Cells are transferred to 96-well microtiter tissue culture plates and cultured until 100% confluency, followed by fixation with glutaraldehyde and storage at −80° C. Intact ECs either on a plate without fixation or in a cell suspension prepared by trypsin treatment are alternative options as EC Ags. Since 1) the inventors previously found the association between elevated AECA and AMR in heart Tx recipients using the fixed/cryo-preserved ECs described herein, and 2) Ags expressed on ECs in suspension may not be identical to those in an adherent form, the inventors decided to use fixed/cryo-preserved ECs on plates. However, the additional options described herein may be used if needed.

For allo-CFC-PBMC, 6 tubes with different conditions are run per sample. 10 μl of anti-CD28/49d and 10 μl of Brefeldin A at 10 μg/ml are added to 1 ml of blood for co-stimulation and prevention of IFNγ secretion, respectively. 100 μl of blood are mixed with 100 μl of donor PBMCs (2×10⁶/ml) or 3^{rd}N PBMC (test conditions), 200 μl of blood alone or mixed with 2 μl of phytohaemaglutinine (PHA, 1 μg/ml) serve as negative and positive controls, respectively, and donor PBMCs (2×10⁶/ml) and 3^{rd}N PBMC alone for background. For allo-CFC-EC, 3 different conditions are run per sample. 200 μl each of whole blood mixed with anti-CD28/49d and Brefeldin A are added to 3 wells on an EC plate; 1^{st} well without EC (negative control), 2^{nd} well with EC (test condition) and 3^{rd} well with EC and 2 μl of PHA (positive control). After 6 hours incubation at 37° C., fixation and permeabilization are carried out. 50 μl of 20 mM EDTA are added to each tube as additional anti-coagulant and incubated for 10 minutes to detach adherent cells. After red blood cells are lysed in FACS Lysis solution, cells are permeabilized followed by staining with an anti-CD3/anti-CD8/anti-CD56/anti-CD16/anti-IFNγ antibody cocktail. All samples are acquired within 24 hours and analyzed with Summit software (Dako Flow Cytometry). IFNγ+ cell number in NK cell subsets, CD3−, CD4+ and CD8+ cells are enumerated and the result is expressed as IFNγ+ cell % in each cell population.

### Example 5

### Methods—Determination of FcγIIIa Genotype by TaqMan SNP Genotyping Assay

Leukocyte pellets stored at −80° C. are resuspended in 200 μl of PBS, submitted for DNA extraction by Qiacube DNA extraction machine, followed by measurement of the DNA concentration by OD₂₆₀, and 20 ng of the genomic DNA are submitted for FcγRIIIa genotype assay.

FcγRIIIa genotype (158V/V, 158V/F or 158F/F) is determined by TaqMan SNP genotyping assay (Applied Biosystems, Carlsbad, Calif.). The assay uses a real time-PCR with TaqMan technology, and consists of 2 primers that amplify all 3 FcγRIIIa genotypes and 2 probes specific to 158V or 158F genotype conjugated with fluorescence FAM or VIC, respectively. FAM- or VIC-probe hybridizes with PCR amplicons from either 158V or 158F genotype, resulting in fluorescence emission from either FAM or VIC, respectively, while both probes hybridize and emit both fluorescence in 158V/F genotype. After PCR, the genotype in each DNA sample is assessed based on the fluorescence intensity emitted from FAM and/or VIC by Applied Biosystems software in 7500 or 7700 real time-thermocycler.

### Example 6

### Methods—Other Lab Tests and Clinical Data

The levels of anti-HLA class I and II antibodies in plasma are quantified by ELISA. Other clinical tests such as flow PRA, anti-HLA antibody specificity, CDC-CMX, flow-CMX, biopsy including C4d staining, sCr levels, donor and recipient CMV and EBV serology, viral-PCR and Cylex T cell immune function assay are performed routinely.

### Example 7

### Methods—Statistical Consideration

Biostatistics Core of CSMC Research Institute is used to provide study planning, database management, and data analysis assistance.

### Example 8

### Methods—Time Frame of Study

Patients are enrolled during the first 17 months. During this period, the samples are processed and analyzed, followed by data entry to the database. Sample collection and analysis without patient enrollment continues from the 18^{th} to the 19^{th} month.

### Example 9

### Methods—Potential Pitfalls

Low EC cell numbers in a well used for allo-CFC-EC is a potential pitfall. Allo-CFC-PBMC uses approximately 2×10⁵ PBMCs per condition as allo-Ags, while approximately 1.5×10⁴ ECs serve as allo-Ags in allo-CFC-EC. However, this number of EC sufficiently activated NK cells as described herein. In case where more EC Ags are required, 48-well EC plates are used that have 3 times larger area per well and approximately 4.5×10⁴ ECs are used per condition. In the instance where more EC Ags are required, fixed or non-fixed EC suspension are used.

### Example 10

### Methods—Availability of Facilities

The Transplant Immunology Laboratory (TIL) is fully equipped for Molecular Biology, Molecular/Cellular Immunology, and Biochemistry/Protein Chemistry. The lab space consists of 2,300 sq. ft., 3 rooms with individualized bench work areas, 2 tissue culture rooms, a dark room, and a fume hood room. Space necessary for this study is available within the TIL. Equipment available for this study include a flow cytometer, tissue culture hoods, incubators, microscopes, table top centrifuges, micro centrifuges, DNA extraction machines, real time-thermocyclers and an ELISA plate reader. Liquid nitrogen tanks, −80° C. and −20° C. freezers, refrigerators for sample and reagent storage are also available. Core facilities provide additional storage as well as other heavy equipment.

Fifteen computers are available. The laboratory features fully networked computers capable of sharing data and information both within the hospital and without. CSMC Enterprise Information Services Department performs maintenance of data and network security, and provides full IT support.

### Example 11

### Assessment of Allo-Sensitization and Prediction of AMR in HS Patients Using Pre-DES/Pre-Tx Whole Blood by Allo-CFC-PBMC

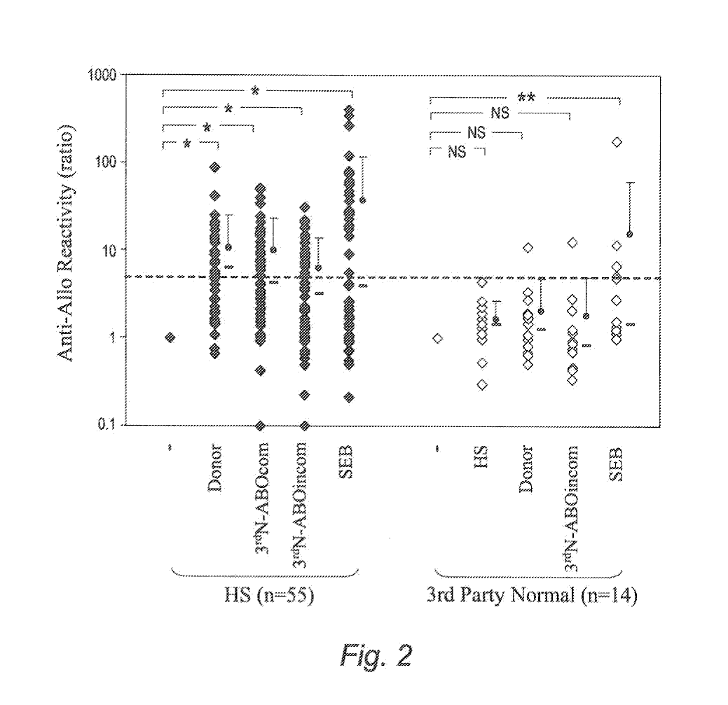

The inventors confirmed the utility of allo-CFC-PBMC for assessment of allo-sensitization and prediction of AMR in HS patients. Anti-allo-reactivity in response to various PBMCs was elevated in many HS patients, while the reactivity in most normal individuals was minimal (FIG. 2). This high reactivity was not against ABO-Ags since elevated reactivity against ABO compatible PBMCs from donor and 3^{rd}N was seen in many HS patients as well.

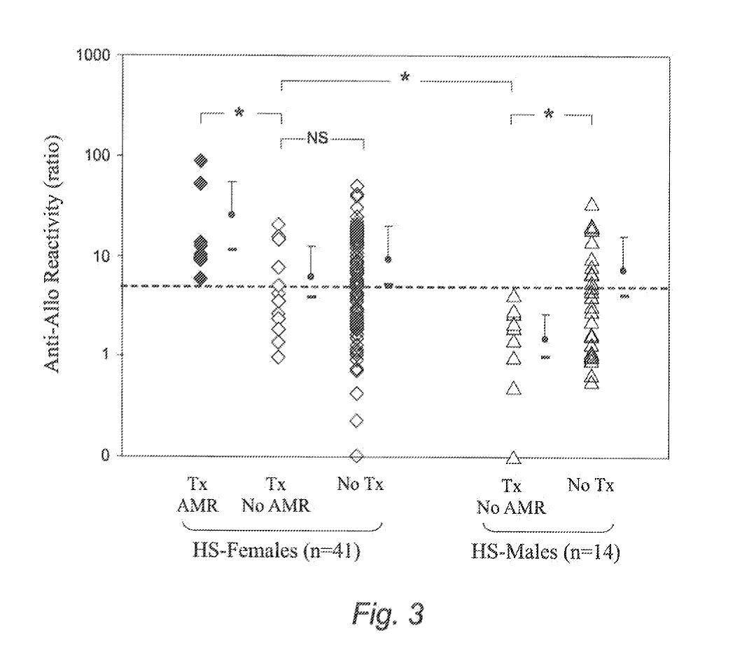

Elevated anti-allo reactivity was seen more in HS females than HS males (FIG. 3), which may explain the higher rate of AMR usually observed in HS females. In fact, all HS males who received Tx showed anti-allo reactivity lower than the cut-off level (<5.0) and none developed AMR. In contrast, many HS females showed high(+) reactivity (>5.0) regardless of transplant status. HS females with AMR showed extremely high(+) reactivity.

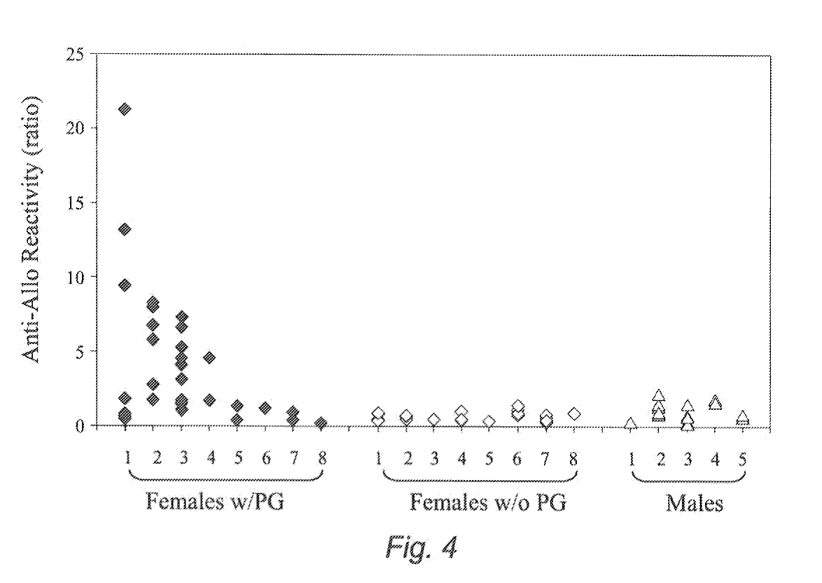

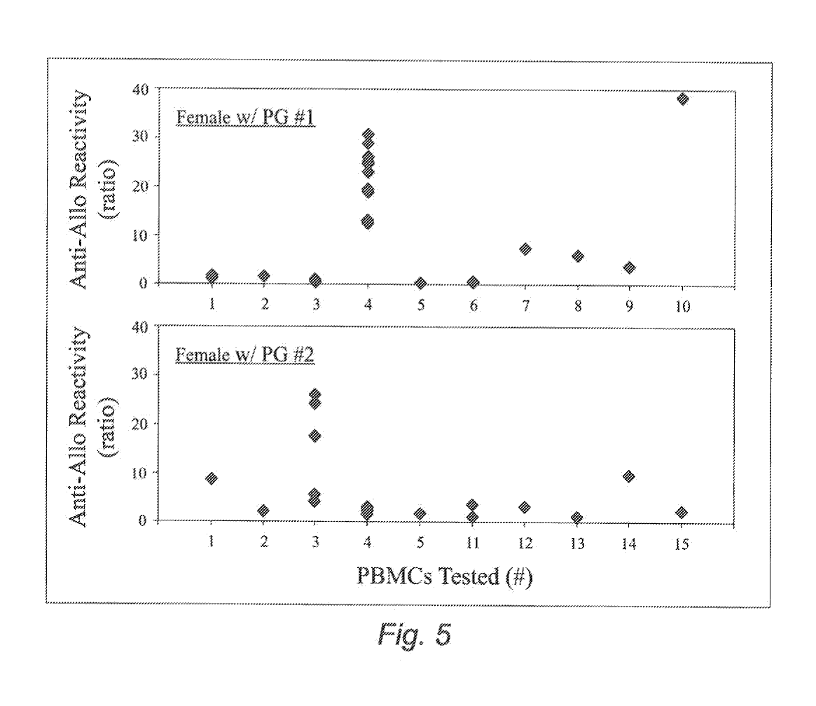

The high reactivity observed in HS females must be due in part to history of pregnancy (PG) since some of the normal females with PG showed elevated anti-allo-reactivity, while the reactivity in all normal females without PG and normal males was minimal (FIG. 4). In addition, the reactivity against the same allo-PBMC tested multiple times over years was fairly consistent (FIG. 5).

Furthermore, these 2 normal females with PG showed high reactivity against PBMCs obtained from their husband, one of their children and 3^{rd}N carrying antigenic pHLA Ags (Table 1). These females also showed antibodies against those antigenic pHLA Ags, although the antibody levels including pHLA-Ag-specific antibody were low in one of these females.

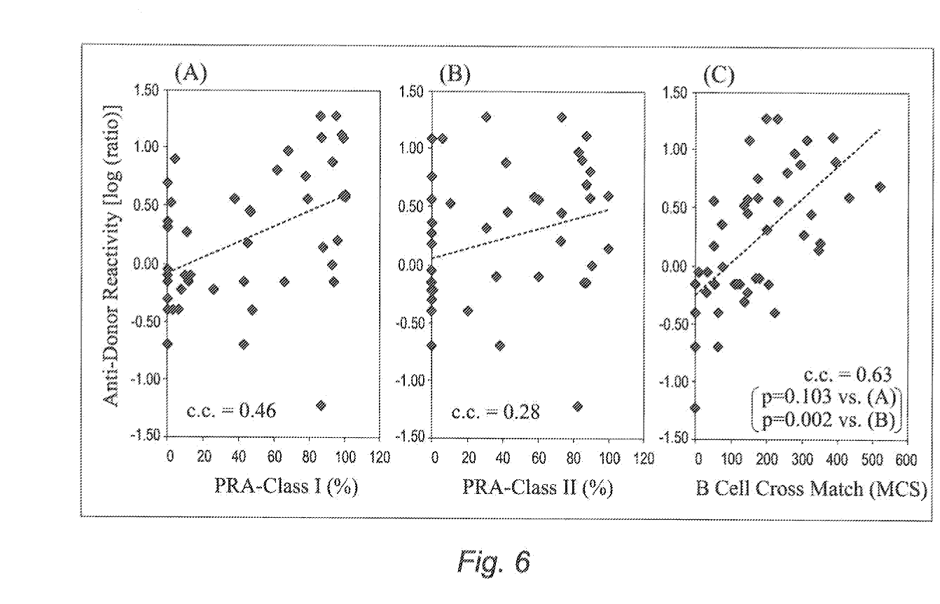

The inventors correlated anti-allo reactivity with anti-HLA Ab levels in HS patients. Anti-donor-reactivity was not significantly correlated with panel reactive antibodies (PRA), non-donor-specific anti-HLA antibody levels, as analyzed by Luminex assay (FIG. 6), while donor-specific CFC reactivity better correlated with donor-specific B cell-CMX, but the correlation was still not that high (c.c.=0.63). A similar trend was found in the above normal females with PG with high(+) CFC reactivity. These results suggest that allo-CFC-PBMC detects allo-sensitization, and is qualitatively associated with presence of anti-HLA antibodies, but not quantitatively so. This suggests that allo-CFC-PBMC is detecting a different aspect of immune reactivity than that seen in antibodies.

### Example 12

### Identification of IFNγ Producing CD3− Cells Detected in Allo-CFC-PBMC

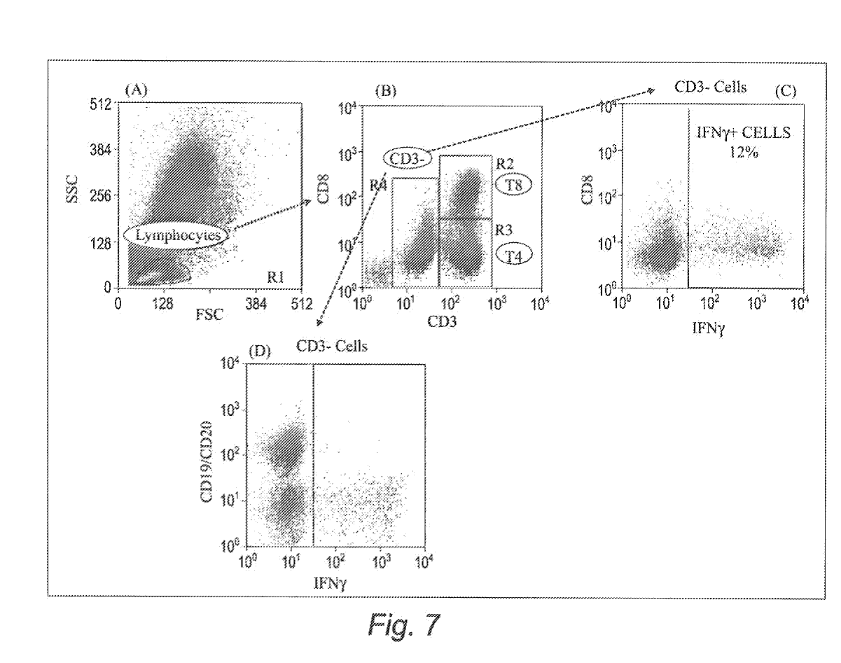

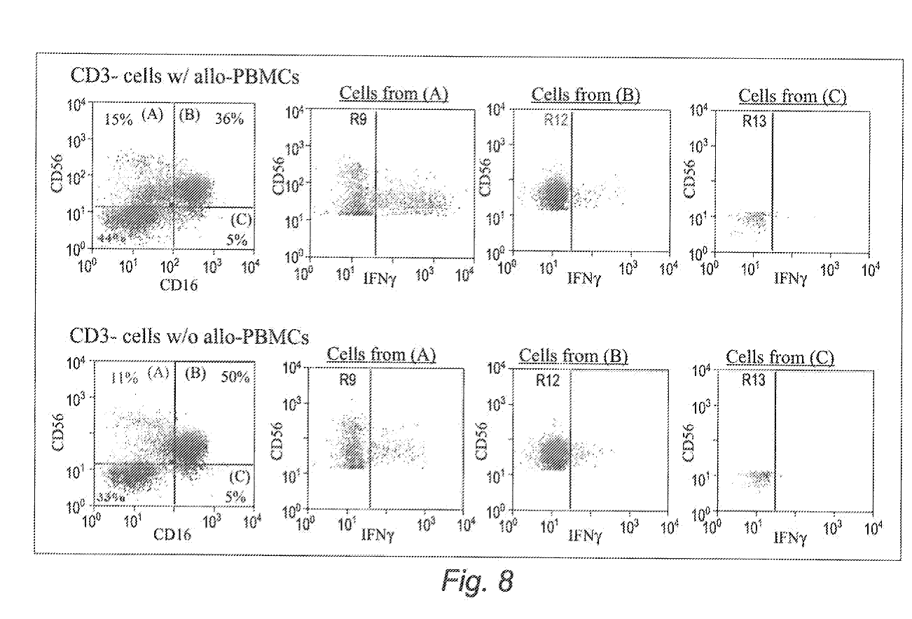

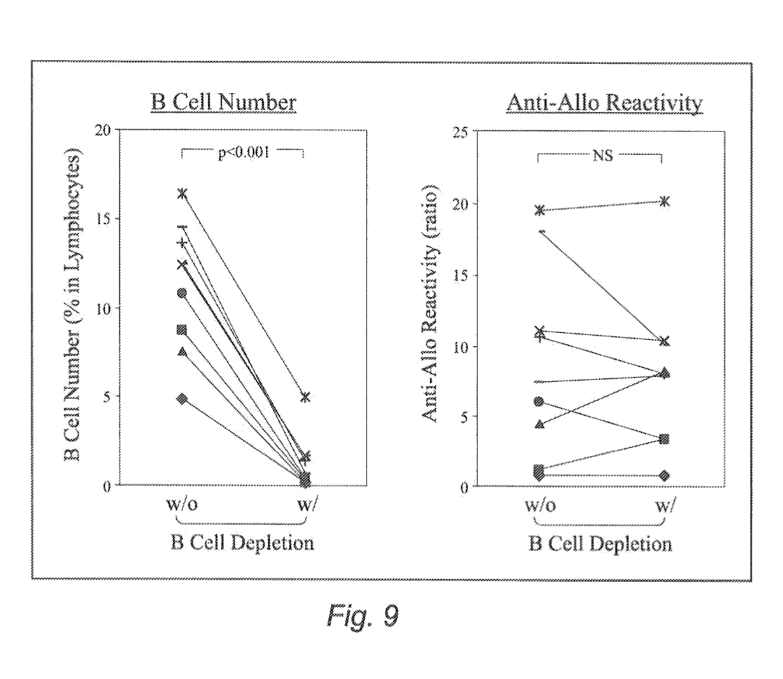

Assessment of sensitization and prediction of risk for AMR in allo-CFC-PBMC is based on detection of IFNγ producing CD3− cells. Identification of these cells is important, since they may be directly involved in the development of AMR and a targeted therapy could be established. The previous allo-CFC-PBMC used a 3-color staining with an anti-CD3/anti-CD8/anti-IFNγ antibody cocktail, and IFNγ+/CD3− cells are enumerated (FIG. 7-A,B,C)). To identify IFNγ+/CD3− cells, the CFC was performed using 6-color staining with additional antibodies to CD19/CD20, CD16 and CD56. IFNγ producing CD3− cells were found to be not CD19+/CD20+ B cells (FIG. 7-D), but CD56+/CD16− NK cells (FIG. 8—upper-A). The inventors also found that IFNγ+/CD56+/CD16−NK cells originated from CD56+/CD16+ NK cells, since CD56+/CD16+ cells significantly decreased (50% to 36%)(FIG. 8—lower-B to upper-B), while IFNγ+/CD56+/CD16− cells significantly increased (11% to 15%)(FIG. 8—lower-A to upper-A) after allo-PBMC exposure. Experiments further show that IFNγ+/CD3− cells detected in allo-CFC-PBMC are NK cells, and not B cells. Despite significant B cell depletion as shown in FIG. 9—left, the anti-allo reactivity did not change (FIG. 9—right).

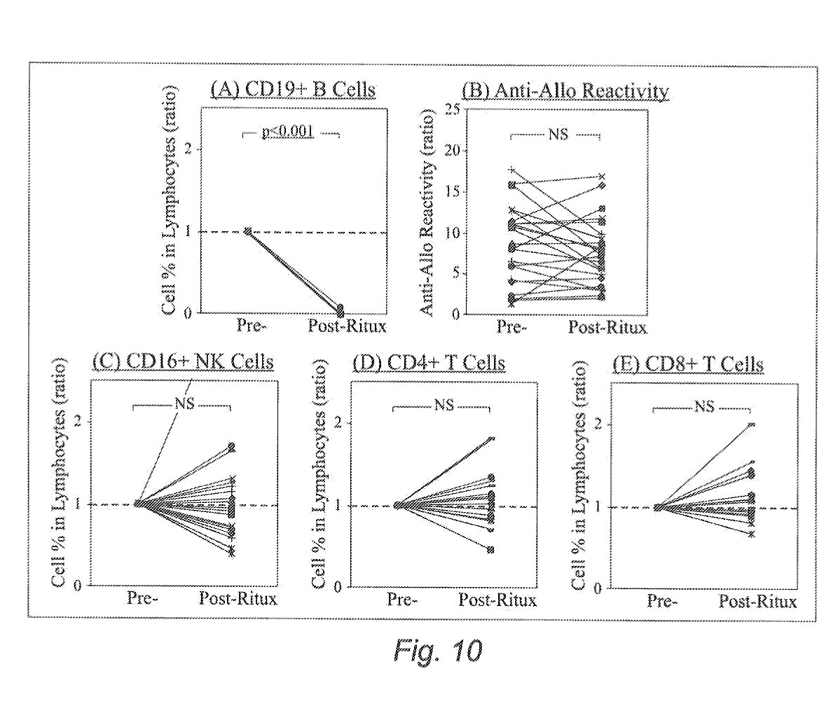

A similar experiment was performed using blood obtained from HS patients pre- and post-IVIG-rituximab-DES. Despite almost complete B cell depletion (FIG. 10-A), anti-allo reactivity did not change in many patients (FIG. 10-B). In contrast to B cell number, other immune cell numbers including NK cells did not change post-DES (FIG. 10-C,D,E). These results again suggest that IFNγ+/CD3− cells detected in allo-CFC-PBMC are unlikely to be B cells.

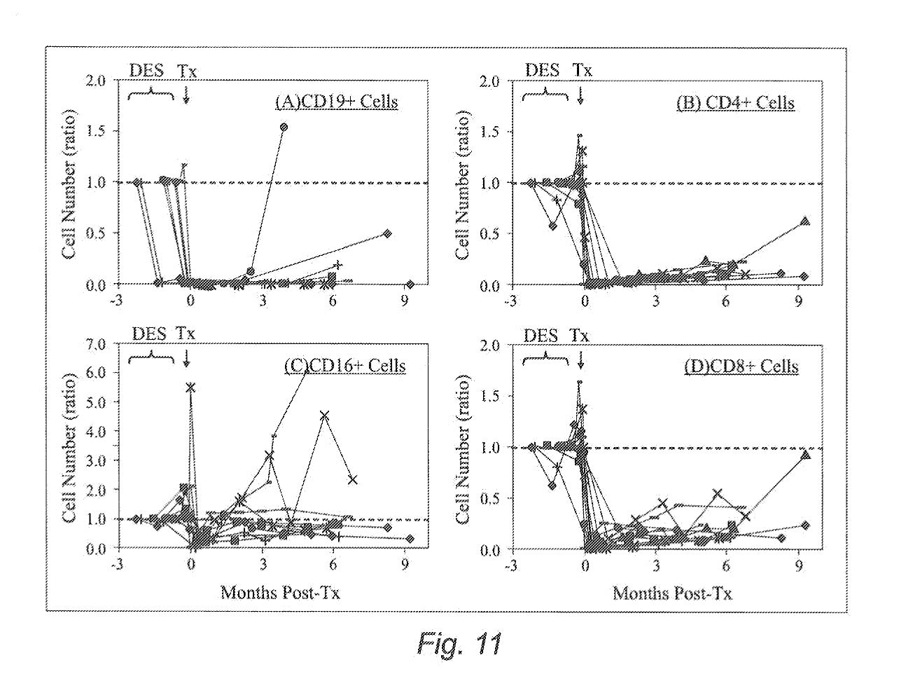

Post-DES, B cell number fell to nearly 0 and B cell depletion persisted post-Tx in many patients (FIG. 11-A). Post-Campath1H induction, CD4+ and CD8+ cell number became significantly lower comparing to pre-Tx levels (FIG. 11-B,D). In contrast, NK cell number only decreased to 50% of pre-Tx levels even during the first 1-2 months post-Tx when AMR often develops (FIG. 11-C). While not wishing to be bound by any particular theory, these results strongly suggest that NK cells may be in part responsible for early onset AMR in HS patients.

### Example 13

### NK Cell Receptor(s) Responsible for NK Cell Activation in Allo-CFC-PBMC

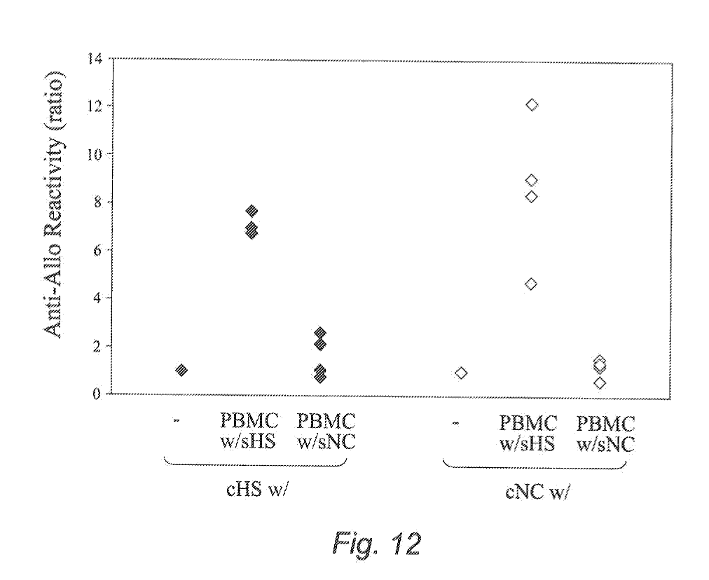

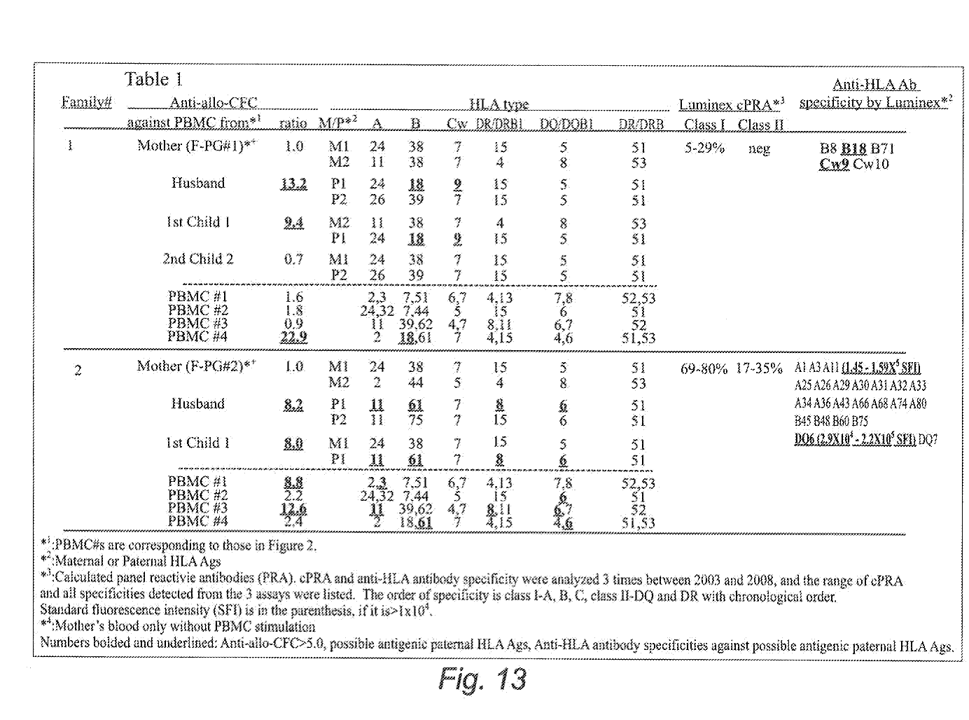

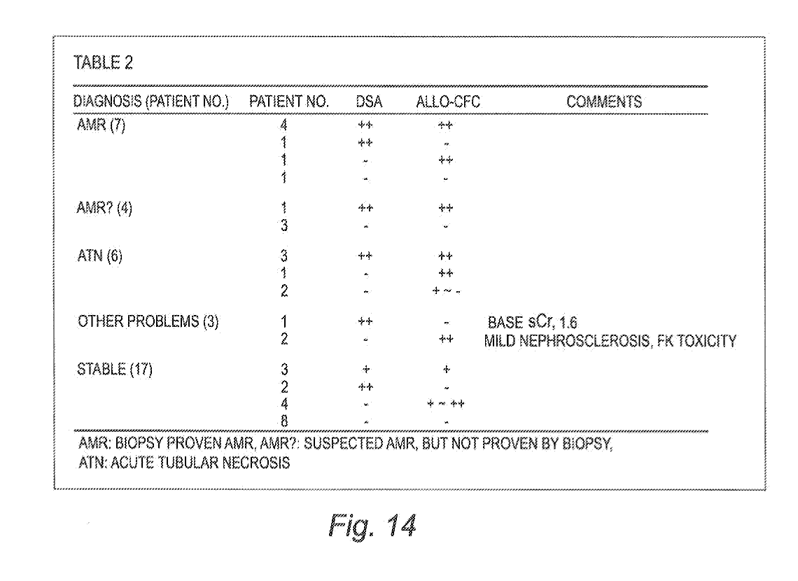

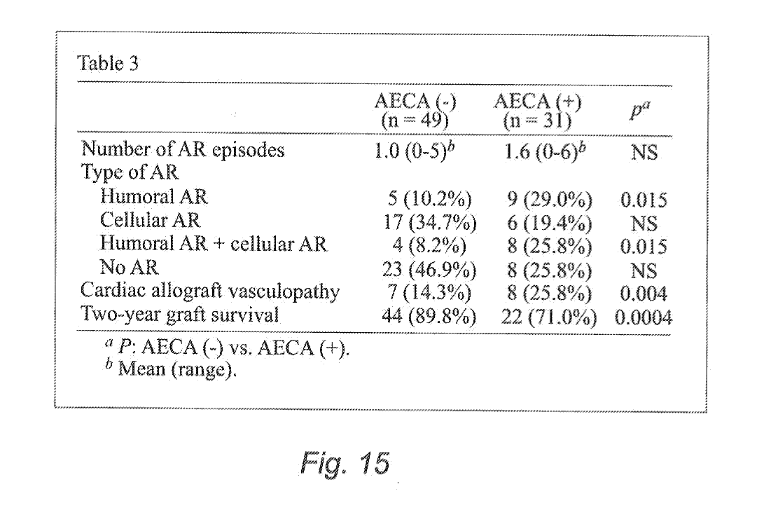

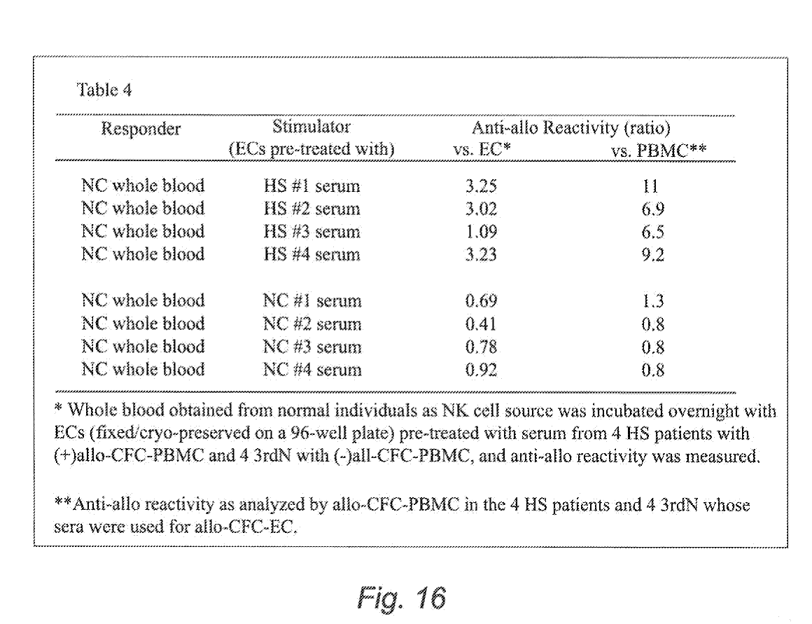

NK cells express various receptors as mentioned in the “Background”. To first know if NK cell activation detected in allo-CFC-PBMC is ADCC or other NK receptor-mediated activation, an experiment was performed (FIG. 12). Both blood cells including NK cells without plasma (cHS, cNC) showed high anti-allo reactivity against the allo-PBMC pretreated with sHS, but not sNC treated allo-PBMC. This result demonstrated that NK cells from either HS patient or 3^{rd}N react with molecules bound to the allo-PBMC pretreated with HS patient serum, but not NC serum, possibly anti-allo-antibodies, resulting in IFNγ production, suggesting that this NK cell activation is very likely to be through ADCC. While not wishing to be bound by any particular theory, this result suggests that some AMR developed in HS patients might be mediated by ADCC, but not complement-mediated cytotoxicity that is diagnosed by C4d deposition on biopsy.

### Example 14

### Monitoring HS Patients Post-Tx by Allo-CFC-PBMC to Predict AMR and Other Episodes

Five of 7 patients with AMR showed high anti-allo reactivity, while 5/7 also showed high DSA (Table 2). Amongst 4 patients with suspected AMR, only one showed high(+) for both DSA and allo-CFC, while the remaining 3 were (−) for both tests, suggesting against AMR in these patients. Interestingly, 4/6 patient showed high anti-allo reactivity at ATN and the remaining 2 patients also showed reactivity elevated from baseline. While not wishing to be bound by any particular theory, it is possible that ATN in these patients may have been caused by ADCC not resulting in C4d deposition. Amongst stable patients, 7/17 showed low(+) anti-allo reactivity, which may warrant follow-up for possible chronic allograft rejection (CAR) in the future. Although allo-CFC-PBMC predicts AMR, 20-30% of patients showed discordant results as shown here. These results suggest that additional factors need consideration to better predict AMR.

### Example 15

### Clinical Significance of AECA after Cardiac Tx

The inventors have previously shown that presence of AECA post-Tx as analyzed by ELISA correlated with greater incidence of AMR, cardiac allograft vasculopathy, and lower 2-year graft survival comparing to AECA(−) cardiac Tx recipients (Table 3).

### Example 16

### Anti-Alto Reactivity as Analyzed by Allo-CFC-EC Assay

In this study, the inventors measure anti-allo reactivity against fixed/cryo-preserved ECs on 96-well plates that were used for studies described herein (Table 3). To confirm the adequacy of the EC plates for use with allo-CFC-EC, a modified allo-CFC-EC was performed as explained in Table 4. Comparing to 4 EC wells treated with 3^{rd}N serum showing ratios <1.0, 3/4 EC wells treated with HS serum showed higher reactivity (>3.0), while 1 well showed minimal reactivity. Based on this result, Ags expressed on fixed/cryo-preserved ECs on 96-well plates are appropriate for use in this study.

### Example 17

### Measurement of FcγRIIIa Genotypes in Normal Individuals by TaqMan SNP Genotyping Assay

FcγRIIIa genotypes in 10 normal individuals were analyzed by TaqMan SNP genotyping assay. The frequency of FcγRIIIa 158VV, VF and FF was 20%, 70% and 10%, respectively, which is slightly different from published data reporting as 47%, 48% and 5%, respectively in 113 individuals (MT3). However, FcγRIIIa 158FF was the least prevalent genotype in accordance with published data. Thus, measurement of FcγRIIIa genotype by TaqMan SNP genotyping is a reliable technique.

### Example 18

### Relevancy of Studies

High rates of AMR remain an obstacle in HS patients receiving DES followed by Tx. Current screening methods for AMR consist primarily of antibody assays, but tools measuring cell-mediated immunity to allo-Ags are lacking. The inventors developed allo-CFC-PBMC to measure allo-Ag-specific CD3− cell response to allo-PBMC. In previous studies, the inventors found: 1) elevated allo-Ag-specific CD3− cells in many HS patients, but not most normal individuals, 2) greater elevation of these cells in HS females than HS males, 3) elevated reactivity in normal females with a history of pregnancy (PG) against paternal HLA (pHLA) Ags, 4) HS patients with high anti-allo-reactivity pre-Tx were at high risk for AMR, and 5) elevated anti-allo-reactivity was often observed prior to or at AMR post-Tx. Based on these findings, the inventors concluded that allo-CFC-PBMC is a novel assay to measure allo-Ag-specific cell responses, can detect allo-sensitization resulting from previous exposure to allo-Ags including PG, and has clinical utility in predicting risk for AMR in HS patients. Additionally, the inventors found IFNγ producing CD3− cells detected in allo-CFC-PBMC were primarily NK cells acting primarily through ADCC, suggesting NK cell involvement in development of AMR.

Although allo-CFC-PBMC can predict AMR, 20-30% of patients showed discordant results (patients with low allo-CFC-PBMC developed AMR and vice versa), suggesting that additional factors need consideration to better predict AMR.

Since 1) ECs on transplanted allografts are primary targets of recipient immune cells, 2) ECs express Ags not expressed on PBMCs, and 3) previous studies have shown the association of elevated pre- or post-Tx anti-EC antibodies (AECAs) with AMR, measurement of anti-allo reactivity against ECs (allo-CFC-EC) might better predict AMR when combined with allo-CFC-PBMC. Therefore, the inventors monitor HS patients using both allo-CFC-PBMC and allo-CFC-EC in this study.

The inventors previously showed that allo-CFC-PBMC is qualitatively, but not quantitatively, associated with presence of anti-HLA antibodies including donor-specific antibody (DSA). Some patients with fairly low levels of DSA developed AMR, others with high levels of DSA do not. Since 1) the inventors found a possible role for NK cells in AMR through ADCC, 2) FcγRIIIa (CD16a) expressed on NK cells plays a central role in ADCC, and 3) FcγRIIIa-158V genotype is known to have higher ADCC activity than FcγRIIIa-158F genotype, the difference in FcγRIIIa genotype might affect graft outcome. Therefore, the inventors will correlate FcγRIIIa genotype with allo-CFC reactivity, AMR or other clinical findings in this study.

In this study, the inventors evaluate the described monitoring protocol to predict acute AMR occurring within 6 months post-Tx. Based on this study results, the inventors investigate the mechanism responsible for CAR, a significant problem in transplantation. A number of studies have implicated HLA or non-HLA antibodies in CAR. The inventors believe that one cause of CAR may be a sub-clinical AMR where NK cells are consistently or periodically activated by allo-Ags with/without involvement of DSA. This could go undetected by currently available tests. In fact, it has been seen patients with negative or low levels of DSA, stable serum creatinine, but (+)allo-CFC-PBMC. These patients are especially of interest for monitoring post-Tx using allo-CFCs to correlate results with CAR episodes.

### Example 19

### Monitoring of NK Cell-Specific Gene Expression to Predict Antibody Mediated Rejection in Kidney Transplant Patients Desensitized with IVIG

High rates of antibody mediated rejection (AMR) remain an obstacle in highly sensitized (HS) patients receiving desensitization (DES) followed by transplant (Tx). Current screening methods for AMR consist primarily of antibody assays. However, tools measuring cell-mediated immunity to allo-antigens (Ags) are lacking. As described herein, inventors developed an intracellular cytokine flow cytometry (CFC) assay to measure CD3− (non-T) cell response to allo-Ags expressed on peripheral blood mononuclear cells (allo-CFC-PBMC) by detecting intracellular gamma-interferon (IFNγ) production. In previous studies, the inventors found: 1) Allo-Ag-specific CD3− cells were elevated in many HS patients and 2) HS patients with high(+) allo-CFC-PBMC pre-Tx were at high risk for AMR, and elevated allo-CFC-PBMC was observed prior to AMR post-Tx in some patients. These suggest the utility of the allo-CFC-PBMC for predicting AMR. Additionally, the inventors found allo-Ag-specific CD3− cells detected in the allo-CFC-PBMC were primarily NK cells. This NK cell response is primarily due to antibody dependent cell cytotoxicity (ADCC), suggesting NK cell involvement in development of AMR.

The inventors performed microarray analysis in the allo-CFC-PBMC setting to identify specific genes expressed in ADCC, and found about 20 ADCC-specific genes including IFNγ. While not wishing to be bound by any particular theory, the inventors believe that: 1) mRNA expression of these genes is elevated in urine of patients who have ADCC in the kidney allograft, resulting in development of AMR, and 2) It will be possible to detect the expression of these genes earlier and more consistently in urine compared to blood since ADCC occurs primarily in the kidney allograft and not systemically. This will allow the earlier and more accurate prediction of AMR.

This study involves a quantitative reverse transcriptase-PCR(RT-PCR) using a real time-PCR with TaqMan methodology to detect mRNA expression of these 20 genes in urine obtained from HS kidney transplant recipients. Results are compared to those from the allo-CFC-PBMC, AMR episodes, biopsy result if available, graft survival and other clinical data. 100 HS patients (living- and deceased-donor Tx patients) included in this study receive DES with IVIG and Rituximab, followed by Tx when flow crossmatch (CMX) is acceptable. Urine samples are collected pre-Tx and at 6 time-points post-Tx (1, 2 weeks, 1, 2, 3 and 6 months), and total RNA extracted from the urine are submitted for the RT-PCRs.

Genes are identified, the expression of which best correlates with and predicts AMR episodes, amongst the 20 genes. Additionally, gene expression results are correlated with allo-CFC-PBMC results, cellular AR, biopsy results, anti-HLA antibody levels and graft survival.

### Example 20

### Development of a Novel Protocol to Predict Risk for Antibody Mediated Rejection by Monitoring Allo-Antigen Response in Patients Undergoing Desensitization with IVIG

The inventors developed a protocol to predict AMR by: 1) instituting allo-CFC using endothelial cells (ECs) as antigenic targets (allo-CFC-EC) since ECs express Ags different from PBMCs, and 2) measuring CD16 (FcγRIIIa) polymorphism since the FcγRIIIa-158V genotype is known to have higher ADCC activity than the FcγRIIIa-158F genotype. Results are compared with allo-CFC-PBMC, AMR episodes, graft survival and other clinical data. 50 HS patients included in this study receive DES with IVIG and Rituximab, followed by Tx when flow crossmatch (CMX) is acceptable. Blood samples are submitted for both allo-CFCs pre-DES, post-DES/pre-Tx and post-Tx (1, 2 weeks, 1, 2, 3 and 6 months). Blood samples pre-DES are tested for CD16 polymorphism. For the allo-CFC-EC, whole blood is incubated overnight with ECs (pooled ECs cultured, fixed and cryo-preserved on 96-well plate obtained from normal individual umbilical vein ECs and/or ECs from donor blood), Brefeldin A and co-stimulators. Intracellular IFNγ in CD3− cells is quantified by flow cytometry. For CD16 polymorphism, DNA extracted from blood is submitted for TaqMan SNP genotyping assay to identify FcγRIIIa-158VV, VF or FF genotype.

The inventors determine whether the combination of the two allo-CFCs along with CD16 polymorphism results in better prediction of AMR. The secondary endpoint is determining whether these results correlate with cellular AR, graft survival, CMX results or anti-HLA antibody levels.

Patients with negative allo-CFCs and FcγRIIIa-FF or VF genotype will generally have AMR-free Tx, those positive for one allo-CFC and FcγRIIIa-VV genotype are likely to develop AMR, and those positive for one allo-CFC and FcγRIIIa-FF or VF genotype are at moderate risk for AMR. This can be applied both pre- and post-Tx, thus providing a critical tool for monitoring patients.

### Example 21

### Background

Herein, the inventors report results from monitoring anti-donor ETC with DSA pre- and post-Tx in a cohort of HS to assess the utility of CFC for predicting risk for AMR and other kidney injury.

### Example 22

### Methods

Pre-/post-Tx blood from 37 HS with living-donor kidney Tx desensitized with IVIG and rituximab followed by Tx were incubated with irradiated donor peripheral blood mononuclear cells. IFNγ+/CD3− cells were enumerated and results expressed as a ratio vs. unstimulated cells. Ratios >3.8 in females, >1.0 in males represent CFC(+). DSA(++) and (+) represent DSA >and <105 SFI, respectively.

Patients: Thirty seven patients (22 females and 17 males) who were desensitized followed by living-donor kidney Tx were included in this study. The time to Tx was 1.4±1.3 months (median 1.1 months) post-desensitization (DES). AMR, CMR and ATN were diagnosed by biopsy.
DES: 2 g/kg of WIG, one month apart with one dose of Rituximab (Ritux) (1 g/dose). Patients who received ABO incompatible Tx were desensitized by one dose of Rituximab, plasmapheresis (PP) 3 to 5 times followed with one dose of WIG.
Sample Collection: Heparinized blood was collected at pre-DES, post-DES, Tx, 1 wk, 2 wk, 1M, 2M, 3M, 4M, 5M, 6M, 9M and 12M post-Tx, and submitted for Anti-donor-CFC.

### Example 23

### Results

Of 37 HS, 16 were biopsied due to increased serum creatinine (sCr) (7AMR, 1CMR, 5ATN [acute tubular necrosis], 3 others). Of 7 with AMR, 6 (86%) showed CFC(+) at time of episode with DSA present in 5/6 (4 DSA[++], 1[+]), 1 HS with CMR showed CFC(−), but DSA(+). 1/7 HS with AMR also had CMR with CFC(+)/DSA(+). All 5 HS with ATN had CFC(+) with 2/5 DSA(++). 1/3 HS with clean biopsies had CFC(+), but DSA(−). Of 21 HS without biopsy, 12 had various complications and 5/12 showed CFC(+) (with femorofemoral bypass surgery, squamous cell carcinoma, bacteremia, appendectomy and presumed AMR). Of the 5, 3 showed DSA(++)/(+) and 1 without DSA data at episode had DSA(+) pre-desensitization. Of the remaining 9 stable HS, 3 were CFC(+). 1 of the 3 HS was DSA(++) and 2 others were DSA(−) post-tx, but 2/3 had DSA(+) pre-desensitization.

### Example 24

### Conclusion

Anti-donor-CFC was (+) in most HS patients 1-2 weeks prior to and/or at AMR with DSA (++), suggesting anti-donor-CFC (+)/DSA (++) post-Tx indicates a risk for imminent AMR. Further, all HS with ATN, some with CMR or some of those without biopsy, but with other inflammation events showed anti-donor-CFC(+), often with DSA present at various levels, suggesting anti-donor CFC (+) might indicate other kidney injuries that might eventually result in graft dysfunction or loss, Patients with (−) anti-donor-CFC and (−) DSA post-Tx are unlikely to develop AMR or other kidney injuries imminently. n a separate study, the inventors showed the majority of IFNγ(+) cells in CFC are NK cells activated via antibody dependent cell cytotoxicity (ADCC). While not wishing to be bound by any one particular theory, these results suggest that detection of IFNγ release in NK cells of HS pre-/post-Tx may be an additional and perhaps independent risk factor for AMR and other allograft dysfunction.

### Example 25

### Cellular Allo Reactivity Against Paternal HLA Antigens in Normal Multiparous Females as Detected by Intracellular Cytokine Flow Cytometry Remains Elevated Over Years Despite Diminution of Anti-HLA Antibody Levels

Sensitization to allo-antigens (Ags) is a significant obstacle to kidney transplantation and risk factor for allograft rejection, especially antibody-mediated rejection (AMR). Desensitization using intravenous immunoglobulin (IVIG) [1-3], plasmapheresis [4,5] and/or chimeric monoclonal anti-CD20 antibodies [6] reduces allo-sensitization and crossmatch (CMX) positivity, facilitating successful transplantation in highly HLA-sensitized (HS) patients. However, allograft rejection (AR), especially AMR, remains a significant obstacle to long-term success. Approximately 28% of desensitized patients who receive a transplant develop acute AMR [7,8]. Several studies have implicated anti-HLA antibodies, especially donor-specific antibodies (DSA), as mediators of early and late AMR that significantly impact long-term graft survival [9,11,43]. Thus, monitoring for and eliminating these antibodies is important for the management of HS patients. However, antibody levels do not always correlate with clinical outcomes [11,12]. The inventors have recently shown that treatment of AMR improved renal function, but patients continued to have high levels of DSA [13]. The same is true in HS patients who are under desensitization treatment and awaiting a transplant. Anti-HLA antibody levels do not always predict the efficacy of desensitization and distinguish patients who will do well from those who experience AMR. Thus, in addition to antibody detection, other specific, novel and non-invasive tests to measure allo- and/or donor-specific immune cell responses and predict the risk for AMR in HS patients during and after desensitization and post-transplant are desirable.

The inventors have previously shown that allo-specific CD3− cell numbers as analyzed by intracellular cytokine flow cytometry (CFC) are elevated in many HS patients, but not most normal individuals [44]. HS females showed higher CFC reactivity than HS males. HS patients with high(+) CFC, especially to donor Ags, may be at high risk for AMR and may need additional pre-transplant desensitization. Although most normal individuals showed minimal reactivity in the CFC assay, the inventors observed that some normal females with one or more previous pregnancies (pPG) showed high CFC reactivity [44]. Allo-sensitization primarily results from previous transplants, blood transfusions and pregnancy [45-49]. While not wishing to be bound by any particular theory, high rates of AMR observed in female HS transplant recipients suggest a significant effect of sensitization through PG on graft outcome. Most previous studies showing allo-sensitization in multiparous females and the effect on graft outcome have been done by detecting antibodies specific to paternal HLA (pHLA) Ags[46,50-53], while only a few studies that detect pHLA-specific immune cell reactivity are reported [54].

The inventors investigated immune cell reactivity against allo-Ags in normal individuals including females with pPG using the CFC assay, determined the utility of the CFC assay to measure sensitization through PG, and determined possible implications of sensitization through PG on transplant outcomes.

### Example 26

### Sample Collection

This study was approved by the Institutional Review Board at Cedars-Sinai Medical Center. Heparinized blood samples were obtained from 21 normal healthy individuals (8 females with pPG [median 2 pregnancies/female, range 1-3 pregnancies], 8 females without pPG, 5 males) multiple times between August 2003 and May 2009, and submitted for the CFC assay. These normal individuals have no history of blood transfusion and transplantation. Serum and acid-citrate-dextrose blood samples were also obtained during this period for anti-HLA antibody tests and HLA typing, respectively. Blood samples from 5 additional normal individuals, family members of the 2 females with pPG, were also tested for various assays. A part of the heparinized blood samples obtained from these normal individuals was used for the preparation of peripheral blood mononuclear cells (PBMCs) used for the CFC assay as stimulator cells (allo-PBMCs).

### Example 27

### Measurement of Intercellular IFNγ Production in Response to All-Ags by the CFC

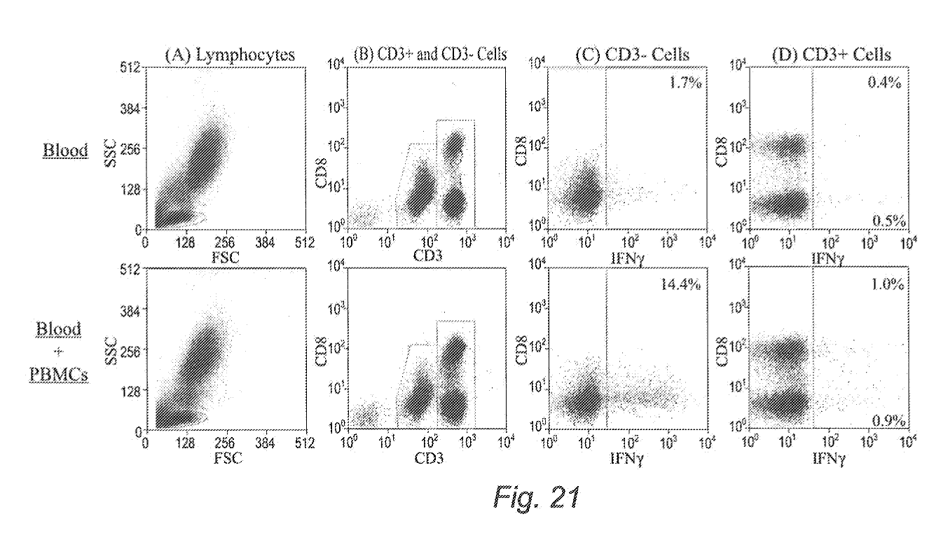

The CFC assay was performed using whole blood as previously described [44]. Briefly, whole blood mixed with CD28/49d and Brefeldin A was incubated with or without irradiated allo-PBMCs overnight. On the following day, after lysing red blood cells followed by permeabilization, cells were stained with fluorescein isothiocyanate-conjugated anti-CD3, tricolor-anti-CD8 and phycoerythrin-anti-IFNγ antibodies, and submitted for flow cytometry. 250,000 cells were acquired per tube in the 1st 80% of the CFC assays and 100,000 cells in the remaining 20% of the CFC assays. Pilot experiments showed no difference in the final CFC results between 250,000 and 100,000 cells used for acquisition. After cell acquisition, lymphocytes first gated by forward/side scatter were further plotted against CD3 and CD8, and IFNγ+ cell % in the CD3− cell population was calculated (FIG. 21). Results of the CFC assay were expressed as the ratio against IFNγ+ cell % in negative control without allo-PBMCs. A ratio N1.0 represents positive reactivity and a ratio N5.0 represents high positive reactivity that may correlate with AMR [44].

### Example 28

### Allo-Ag Preparation

PBMCs were isolated from heparinized blood obtained from normal individuals by density-gradient centrifugation using Ficoll-Hypaque as previously published [40]. PBMCs were aliquoted and frozen. Either freshly isolated or frozen PBMCs were used for the CFC. Pilot experiments showed no difference in stimulation between fresh and cryopreserved PBMCs. Immediately prior to testing, PBMCs were irradiated at 2500 rad.

### Example 29

### HLA Typing

The HLA typing has been previously described [13]. Briefly, intermediate resolution class I and class II HLA typings were performed by a sequence specific oligonucleotide probe method according to the manufacturer's guidelines (One Lambda, Inc., Canoga Park, Calif.).

### Example 30

### Panel Reactive Antibodies (PRA), Anti-HLA Antibody Specificity, Anti-HLA Class I and Class II Antibody ELISAs

PRA and antibody specificity assays have been previously described [13]. Briefly, the binding level of HLA-specific antibodies was determined by the multianalyte bead assay performed on the Luminex platform. The single antigen Luminex bead assay was standardized with Quantiplex beads (One Lambda, Inc., Canoga Park, Calif.) and results were expressed as standard fluorescenceintensity (SFI). Final specificity analysis was analyzed through HLA Visual 2.2 software (One Lambda, Inc., Canoga Park, Calif.). The antigen frequency PRA was calculated based on the frequency of antigens detected with SFIN20,000 in the Luminex single bead assay and their combined antigen frequency in over 54,000 donors in the UNOS database (mTilda, Outland Enterprises, Eugene, Oreg.). Anti-HLA class I and class II IgG antibodies were quantified by Lambda Antigen Tray™ Mixed Class I & II ELISA kit (One Lambda, Canoga Park, Calif.) as previously published [44]. Briefly, serum anti-HLA class I or class II IgG antibodies bound to a mixture of affinity-purified HLA class I or class II Ags coated on an ELISA plate were detected by alkaline-phosphatase-conjugated anti-human IgG followed by addition of the substrate. The OD multiplied by 1000 was used as antibody levels (units). When the OD was higher than that of the positive HLA serum control, the serum was diluted with 0.1% BSA-PBS by up to 1000 times and reanalyzed. Plasma samples obtained from heparinized blood were also used for anti-HLA antibody ELISA. No difference in antibody levels between serum and plasma was noted in pilot experiments. Anti-HLA class I and class II antibody levels in 8 females without pPG (previous pregnancy) and 5 males included in this study were 190±68 and 150±39 units, respectively, and the high normal limit was calculated as 302 and 214 units (mean+1.65 SD), respectively. Antibody levels obtained from females with pPG were excluded from this calculation since some of females with pPG showed significantly higher anti-HLA antibody levels compared to females without pPG and males.

### Example 31

### Results

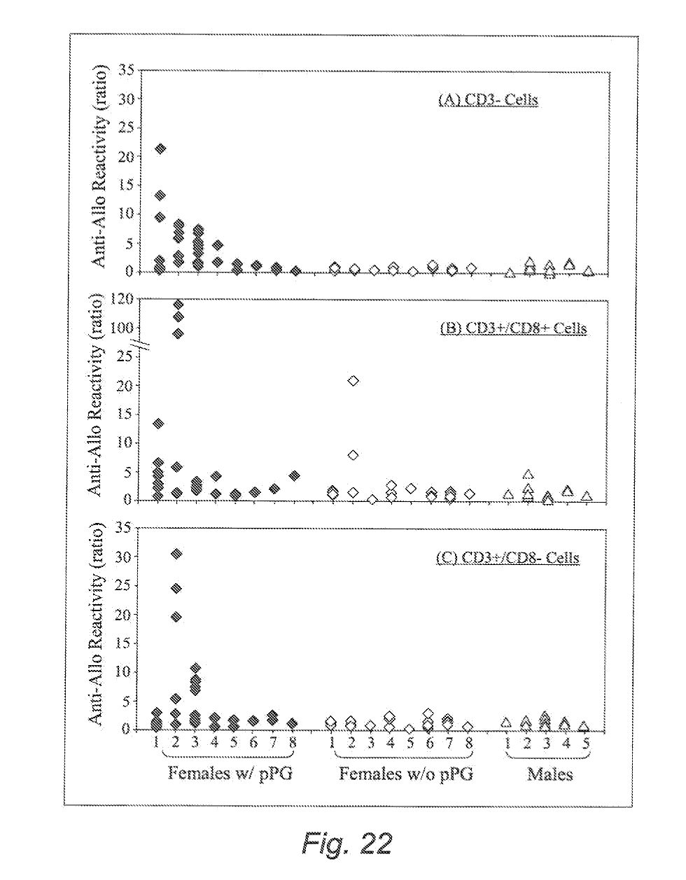

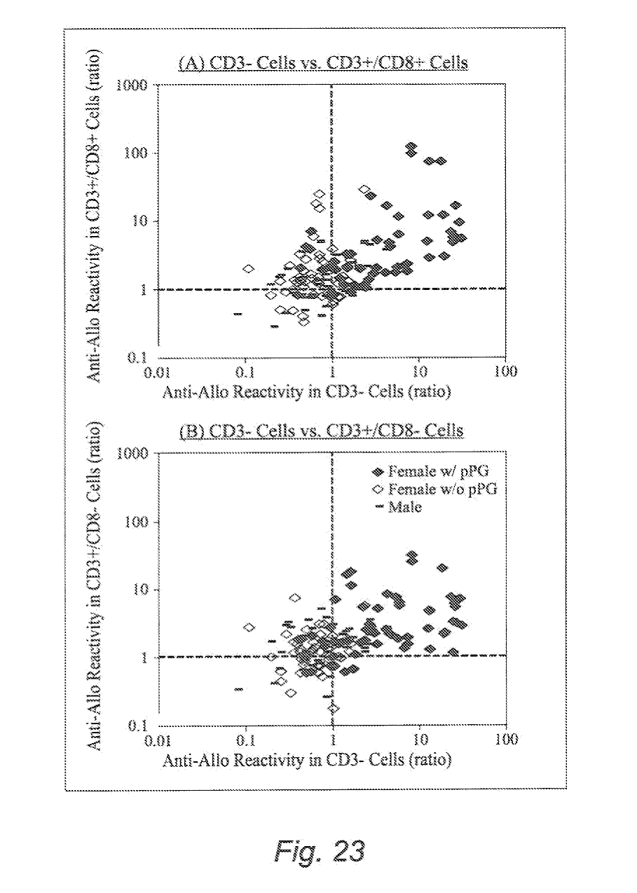

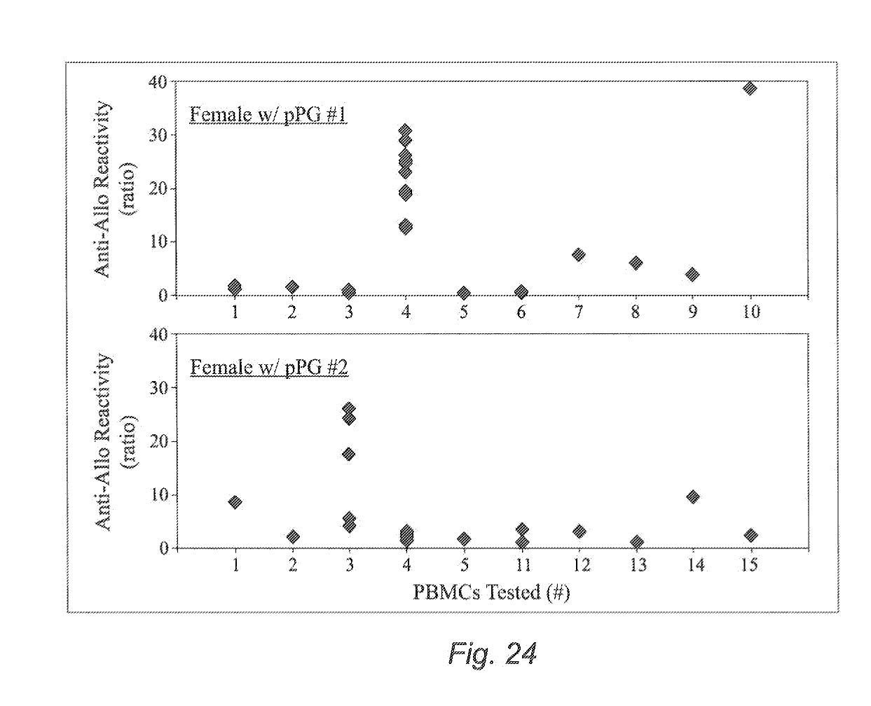

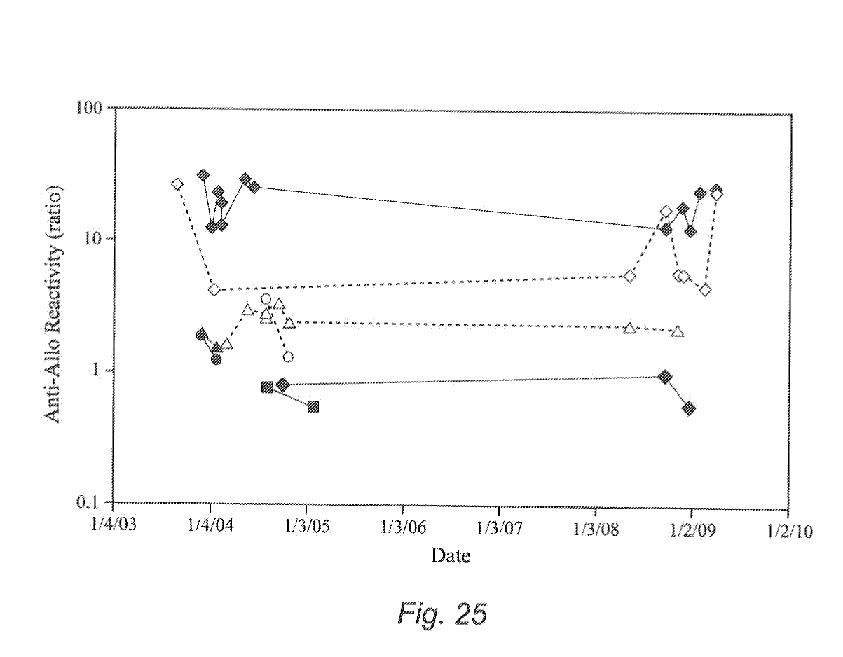

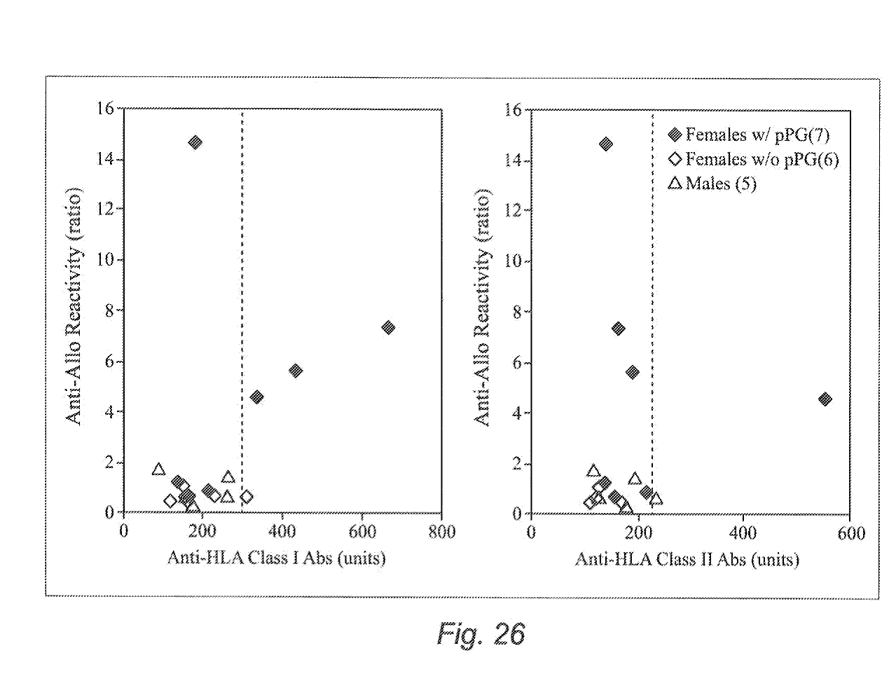

A typical picture of the CFC assay result is shown in FIG. 21. Anti-allo reactivity in CD3−, CD3+/CD8+ and CD3+/CD8− cells in response to various PBMCs in normal females with pPG, females without pPG and normal males is shown in FIG. 22. Four of 8 females with pPG (#1-4) showed elevated reactivity in CD3− cells (A) against at least one of PBMCs tested (average ratio 4.7±1.4), while the remaining 4 (#5-8, 0.76±0.42), 8 females without pPG (0.65±0.24) and 5 males (0.99±0.59) showed minimal reactivity to all PBMCs tested. In contrast, 4 of 8 females with pPG (#1, 2, 4 and 8) showed elevated reactivity in CD3+/CD8+ cells (B), while one of each in females without pPG and males also showed elevated reactivity. Only 2 of the 8 females with pPG (#2 and 3) showed elevated reactivity in CD3+/CD8− cells. Elevated reactivity was not consistently observed in these 3 cell populations in the same individual. FIG. 23 showed anti-allo reactivity in CD3− cells vs. CD3+/CD8+ or CD3+/CD8− cells in each of 158 CFC assay results available. There was a trend of correlation between the two, especially between CD3− and CD3+/CD8+ cells. However, blood with high reactivity in CD3− cells showed a wide range of reactivity in the other 2 cell populations, and some of those with low reactivity in CD3− cells showed high reactivity in the other 2 cell populations. Since this inconsistent reactivity amongst these 3 cell populations was also observed in HS patients and the reactivity in CD3+ cells detected in HS patients did not correlate with AMR episodes as previously reported [44], we focused only on anti-allo reactivity in CD3− cell population for further analysis. Since each data point shown in FIG. 22 is the average of multiple CFC results against each PBMC, individual reactivity against each PBMC in 2 females with pPG (#1 and #2) is shown in FIG. 24. Both females showed high, moderate and minimal reactivities to various PBMCs. The reactivities against the same PBMCs remained consistent over multiple tests (FIG. 24) performed over a period of up to 5 years (FIG. 25). The inventors next determined if anti-allo reactivity is associated with anti-HLA antibody levels as analyzed by ELISA that represents total anti-HLA class I or II antibody levels. Some females with pPG showed high reactivity despite low anti-HLA antibody levels, while the remaining individuals without elevated anti-allo reactivity all showed negative anti-HLA antibody levels (FIG. 26).

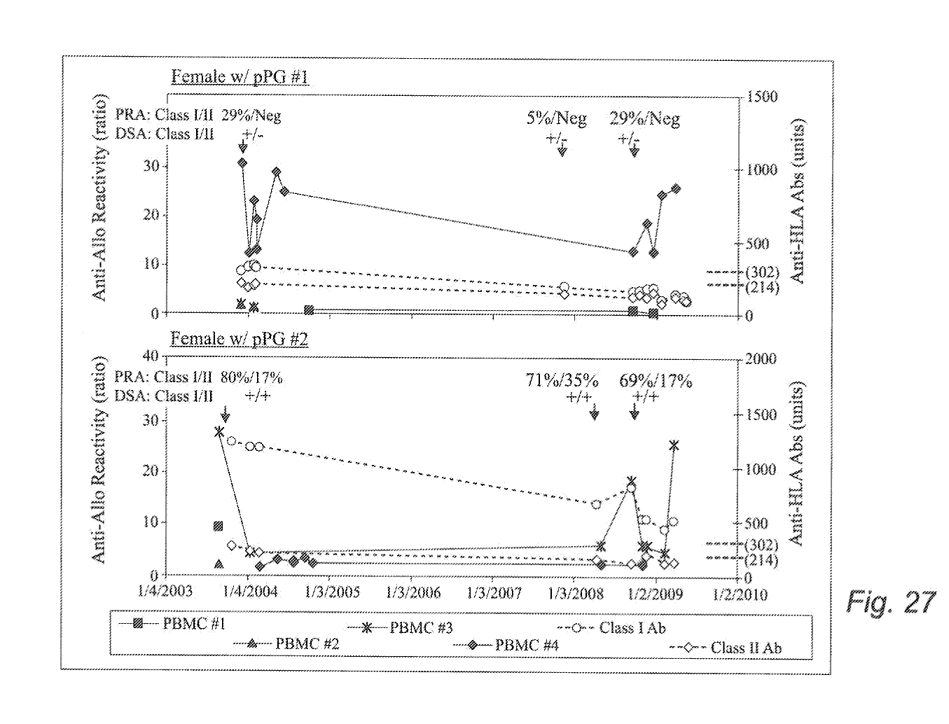

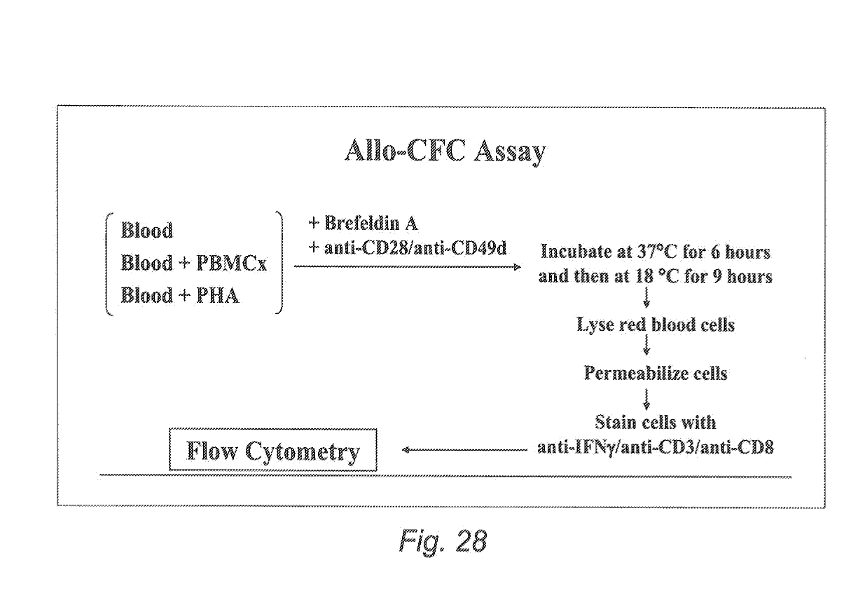

Since the above 2 females with pPG (#1 and #2) had no histories of blood transfusion or transplantation, the inventors hypothesized that the high reactivity to specific PBMCs must be due to allo-sensitization to pHLA-Ags through PG. Thus, we next examined the reactivity against PBMCs obtained from the husbands and children. As expected, both females #1 and #2 showed high reactivities to PBMCs from their husbands (ratio 13.2 and 8.2, respectively) and one of their children (9.4 and 8.0, respectively) (FIG. 13). Female #1 showed minimal reactivity to PBMCs from the 2^{nd} child (0.7). Since both females showed high reactivities to PBMCs from the 1st child, the possible antigenic pHLA-Ags must be B18 and Cw9 in female #1, and A11, B61, DR8 and DQ6 in female #2, and the remaining HLA-Ags are common to themselves. Female #1 showed high anti-allo reactivity (22.9) to PBMC #4 carrying B18, while she showed minimal reactivity to PBMCs #1-3 that did not have those pHLA-Ags. Female #2 showed high reactivity to PBMCs #1 and #3 (8.8 and 12.6, respectively) which carry antigenic pHLA-Ags A11 and A3 which shares epitopes with A11. There was only minimal to moderate reactivity against PBMCs #2 and #4 (2.2 and 2.4, respectively) even though those PBMCs also carried some of those pHLA-Ags, B61 and DQ6. These 2 females with pPG also were positive for anti-HLA antibodies in their serum. Female #1 showed fairly low anti-HLA antibody levels as analyzed by ELISA (97-335 units) and Luminex PRA assay (class I: 5-29%, class II: negative), While female #2 consistently showed high anti-HLA antibodies especially anti-HLA class I antibody (435-1223 units) although the levels fluctuated with multiple tests spanning a period of over 5 years, and PRA (class I: 69-80%, class II: 17-35%) (FIG. 13, FIG. 27). However, both females had antibodies specific to antigenic pHLA-Ags and other Ags as well. The levels of some antibodies (A11 and DQ6) in female #2 were very high (N1×105 SFI).

### Example 32

### Discussion

The inventors demonstrated that elevated allo-Ag-specific CD3− cells as analyzed by the CFC were detected only in normal females with pPG but not in normal females without pPG or normal males who have no history of blood transfusion and transplantation. The anti-allo reactivity was against pHLA-Ags and the reactivity against the same PBMC was fairly consistent over months and years, suggesting that the CFC detects sensitization against allo-Ags resulting from PG. It is known that pregnancy can result in HLA-sensitization against pHLA-Ags expressed in fetal tissues. This has been shown by detection of antibodies specific to pHLA-Ags in multiparous females primarily by complement-dependent cell cytotoxicity assays [45-47]. In this study, the inventors also detected pHLA-specific anti-HLA antibodies by Luminex single-HLA-Ag assay in 2 multiparous females who showed (+) reactivity in the CFC (FIG. 13). Female with pPG #1 showed fairly low levels of anti-HLA antibodies over the years as analyzed by ELISA (normal range) and Luminex cPRA (class I: 5-29%, class II: negative) (FIG. 13 and FIG. 25), but antibodies specific to those pHLA-Ags, B18 and Cw9, were consistently detected. The remaining antibodies detected against B8, B71 and Cw10, are known to share epitope(s) with B18 and Cw9 [55,56]. Despite low anti-HLA antibody levels, she consistently showed very high CFC reactivity against PBMCs from her husband, one of her children and a 3rd party carrying pHLA-Ags. The individual with pPG #2 showed high anti-HLA antibody levels with various specificities as detected by ELISA (500-1300 units) and Luminex (class I: 69-80%, class II: 17-35%). Among those antibodies, those to pHLA-Ags, A11 and DQ6, were N1×105 SFI, a level which has been reported to be associated with high risk for AMR in HS patients [13]. Other specificities detected in her blood were also known to share epitopes with the pHLA-Ags A11, B61 and/or DQ6 [55,56]. Although her reactivity in the CFC fluctuated over the 5 years, it was always in the high range (FIG. 25). Anti-HLA antibody levels in other individuals with minimal CFC reactivity were consistently negative or minimal (FIG. 24). While not wishing to be bound by any particular theory, these results suggest that the CFC detects allo-sensitization, and is qualitatively associated with presence of anti-HLA antibodies, but not quantitatively so. This suggests that the CFC is detecting a different and possibly complementary aspect of immune reactivity than that seen in antibodies.

Detection or quantification of anti-HLA antibodies is currently the gold standard to assess HLA-Ag exposure and the degree of allosensitization. However, tools to assess HLA sensitization by measuring HLA-Ag-specific immune cell reactivity are few. Van Kampen et al. [45,54] have shown that primed cytotoxic T cells (CTLs) specific for inherited pHLA-Ags were found using limiting dilution analysis in women who had formed anti-HLA antibodies and these primed CTLs can persist for more than 10 years after PG. These primed CTLs were found more frequently in women with persisting allo-antibodies, but can still be detected when the antibodies have disappeared, suggesting the utility of this cellular test for detection of HLA-sensitization in women with a history of PG awaiting transplantation. In the inventors allo-CFC assay, whole blood to be tested was incubated with irradiated stimulator PBMCs overnight, followed by measurement of IFNγ+CD3− cells by flow cytometry. Both studies showed elevated number of immune cells reactive with allo-Ags (CTL in their study and CD3− cells in our study) in some females with pPG and HS patients awaiting transplantation [44, 50-53, 14]. In the inventor's CFC, CD3+ T cells reactive with allo-Ags can be detected as well. However, unlike IFNγ+CD3− cells, elevated IFNγ+CD3+ T cells in response to allo-PBMCs were not consistently detected in HS patients [44] or females with pPG (FIGS. 22 and 23). While not wishing to be bound by any particular theory, it is possible that immune cells with cytolytic activity in response to allo-Ags detected in Van Kampen's study may also be CD3− cells since CTLs in their study were not identified as CD3+ cells. While not wishing to be bound by any one particular theory, preliminary results indicate that the primary IFNγ producing cells in the CFC are NK cells, and not B cells. NK cells have recently been found to have Ag-specific adaptive immune features [57,32]. This was also suggested by the inventors findings of persistence of elevated IFNγ+CD3− cells in the CFC after B cell depletion with Rituximab (data not shown). While not wishing to be bound by any particular theory, the NK cell IFNγ production detectable by CFC may implicate NK cells as participants in adaptive immunologic memory and AMR, and point the way to more specific therapies. HLA-sensitization including that through PG is thought to be a risk factor for AMR in HS patients [50-53,14]. Higher rates of AMR seen in HS females might be in part reflected by this. Most previous studies have demonstrated that sensitization to inherited pHLA-Ags induced during PG resulted in unfavorable transplant outcome when the female receives an allograft carrying the pHLA-Ags [50-53]. In a previous study [44], the inventors have shown that HS females showed higher CFC reactivity than HS males and all HS patients who developed AMR in the study were females showing extremely high CFC reactivity. Based on this, the inventors suggested HS patients with high(+) CFC, especially to donor Ags, may be at high risk for AMR and may need additional pre-transplant desensitization. Van Kampen et al. have reported the association of presence of donor-HLA-Ag-specific CTLs and early rejection or graft loss in HS patients using limiting dilution assay [14], suggesting that the assay is an additional tool to identify HS patients for successful transplant. Mulder et al. [15] detected anti-HLA producing B cells in vitro in PBMCs from multiparous women with serum anti-HLA antibodies using a CD40L culture system and HLA-A2 tetramers in a subsequent study [16]. Zachary et al, have recently reported the frequencies of HLA-specific B cells were significantly higher among sensitized than non-sensitized patients using HLA-A2, A24 and B7 tetramers loaded with HIV peptides [17], suggesting the utility of tetramer staining for assessing the risk for AMR. However, the correlation between the frequency of HLA-specific B cells and AMR episodes was not shown by either investigator. Involvement of B cells in AMR in HS patients is still unclear at present.

Poggio et al. [18] and Korin et al. [19] have shown the utility of pre- and post-transplant monitorings of allo-reactive T cells to assess risk for allograft rejection in kidney transplant patients using ELISPOT and CFC, respectively. However, patients included in those studies were primarily non-HS patients, and allograft rejections observed in their studies were primarily CMR with AMR observed in a few patients. Although mothers are exposed to all pHLA-Ags during PG, it does not appear that they become sensitized to all pHLA-Ags. Female with pPG #1 in this study showed high CFC reactivity to PBMCs from the 1^{st} child who carries the 1st set of pHLA, but not PBMCs from the 2^{nd} child who inherited the 2nd set of pHLA, and anti-HLA antibodies detected in her blood were also specific to the 1st set of pHLA-Ags. It is not clear why she was sensitized to the 1st set of pHLA, but not the 2nd set of pHLA. Dankers et al. have reported that 1) mothers with particular HLA-Ags were more immunized comparing to those expressing other HLA-Ags, 2) certain HLA mismatches of their children are more immunogenic than other mismatches, and 3) the immunogenicity of particular HLA mismatches is dependent on the HLA phenotype of the mother [58]. In female with pPG #2, the possible antigenic pHLA-Ags were A11, B61, DR8 and DQ6, since she showed high CFC reactivity to PBMCs from her husband and her child, and antibody specificities to those Ags and other Ags sharing epitopes with those were detected. However, she showed high reactivity to PBMC from 3rd individuals who carry A3, A11 and DR8, while her reactivity to PBMC carrying B61 and/or DQ6 was low-moderate, despite high levels of anti-DQ6 antibody (N1×105 SFI). In this study, both females with pPG were sensitized to pHLA-Ags through the 1st pregnancy. Although it is thought that multiple pregnancies are a higher risk for sensitization, one pregnancy is sufficient to be sensitized to HLA in some females. While not wishing to be bound by one particular theory, this suggests that any exposure to HLA or alto-Ags such as through PG, blood transfusion or transplantation is a risk factor for sensitization and must be taken into consideration for assessment of sensitization.

### Example 33

### IFNγ Production by NK Cells after Allo-Antigen Exposure

The inventors have shown that allo-Ag-specific CD3− cell number analyzed by intracellular cytokine (IFNγ) flow cytometry (CFC) are elevated in HS pre-Tx and often with AMR episodes. The inventors also found IFNγ+/CD3− cells in CFC were primarily NK cells, suggesting a possible role in AMR in HS. As described herein, the inventors report the mechanism(s) responsible for NK cell activation in CFC and their association with post-Tx biopsy findings.

### Example 34

### Studies Performed

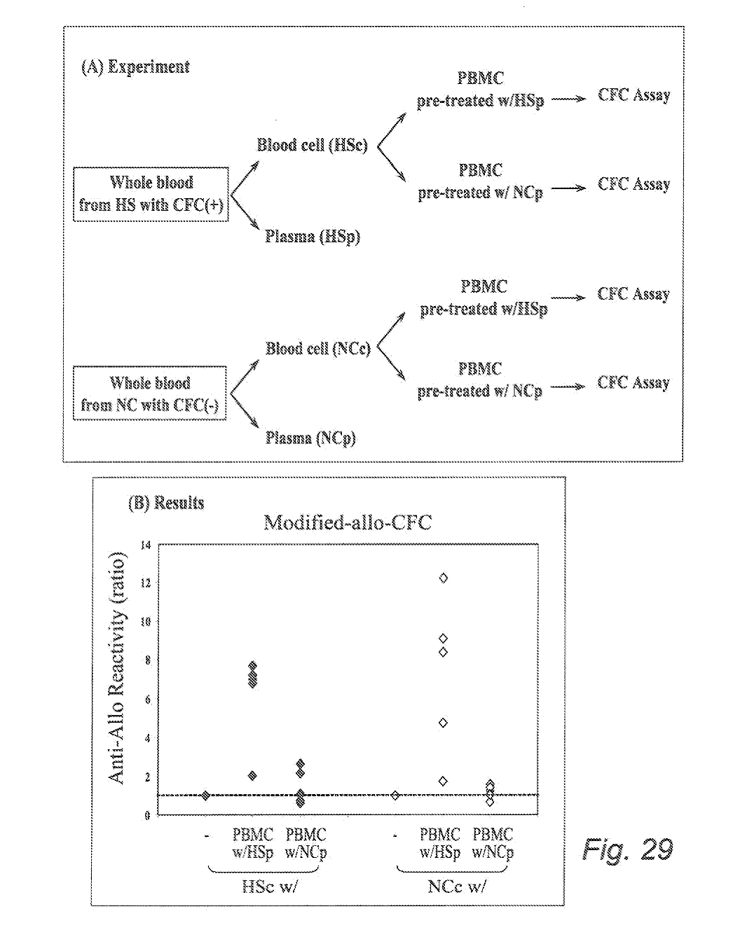

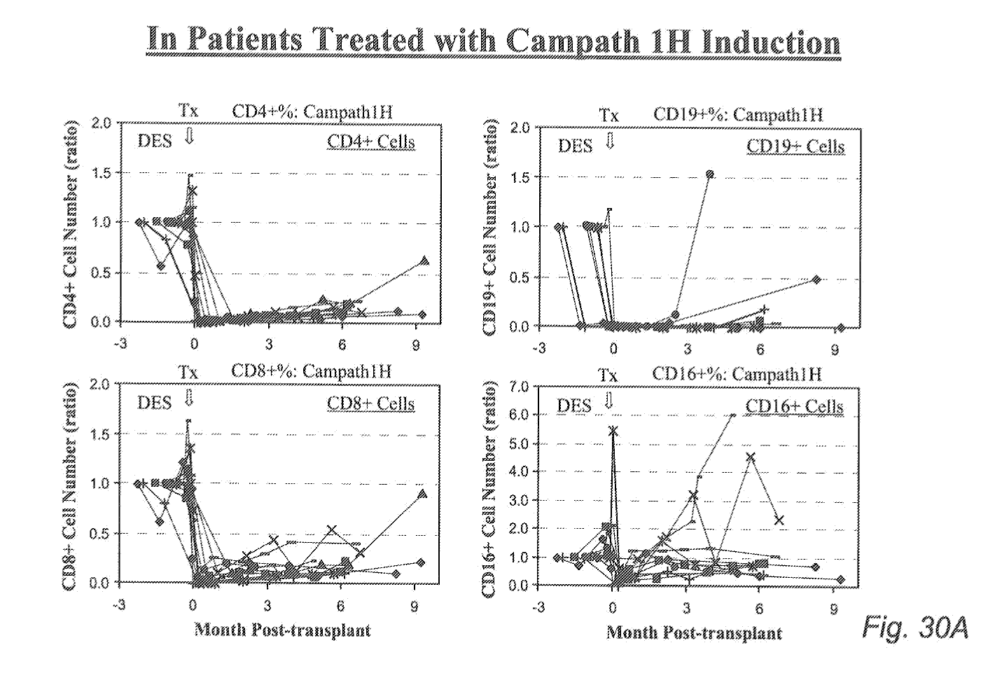

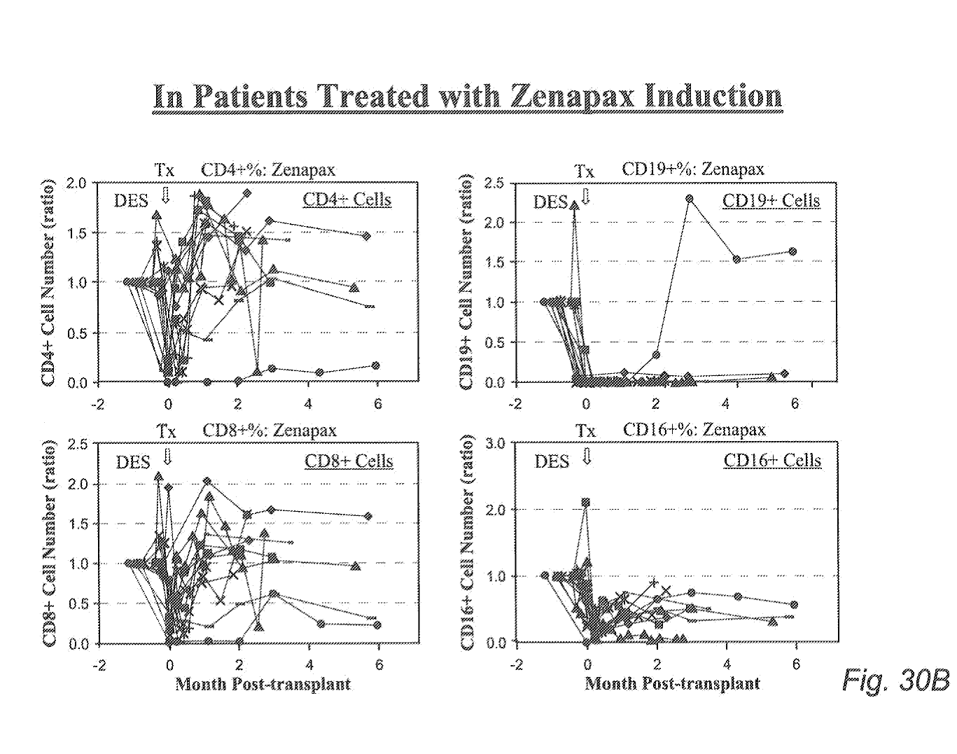

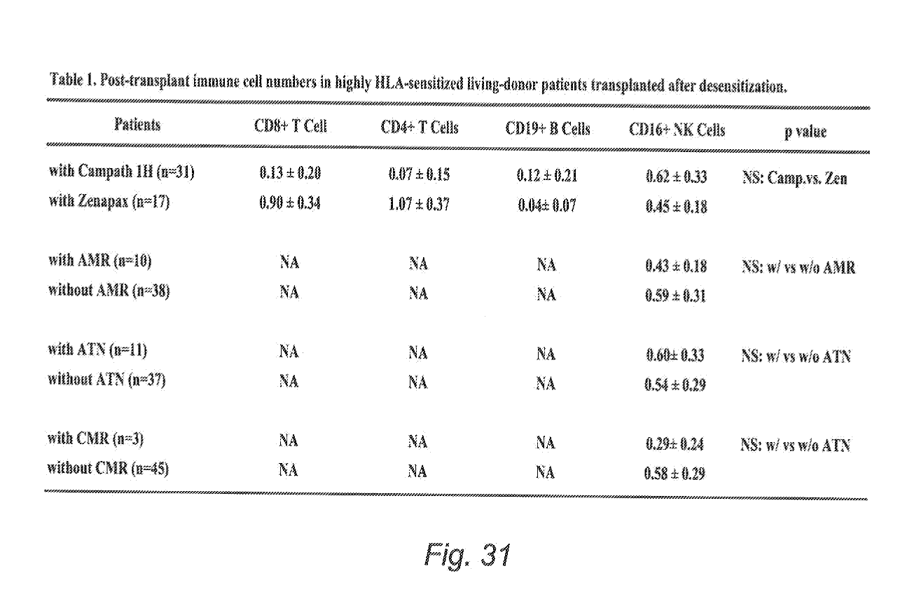

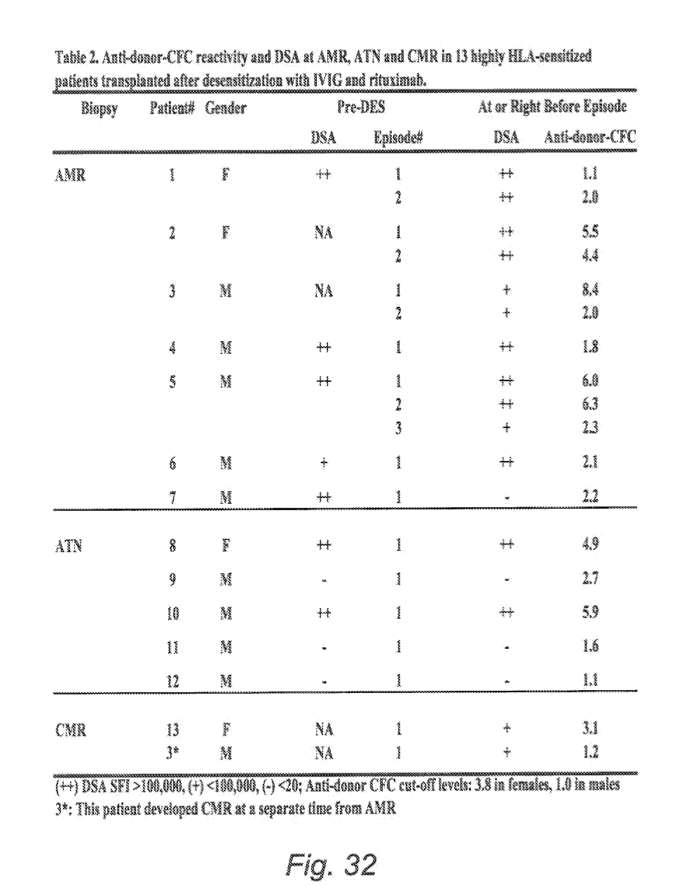

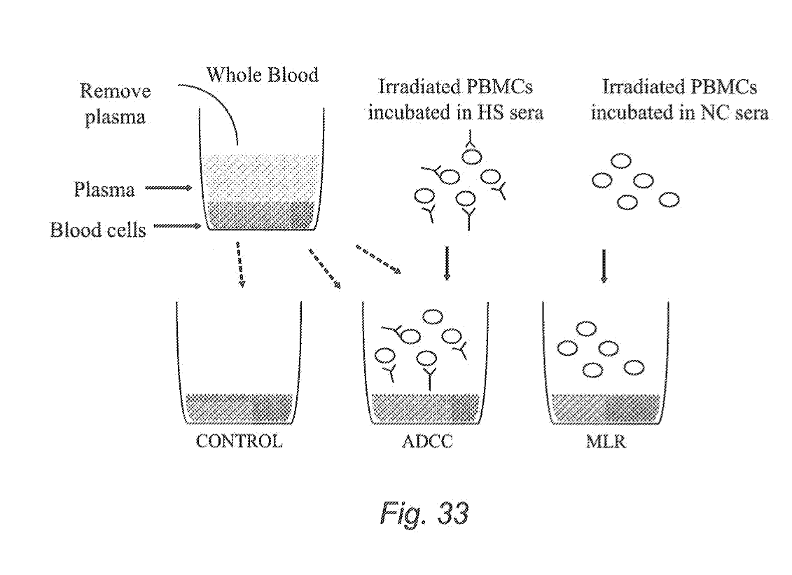

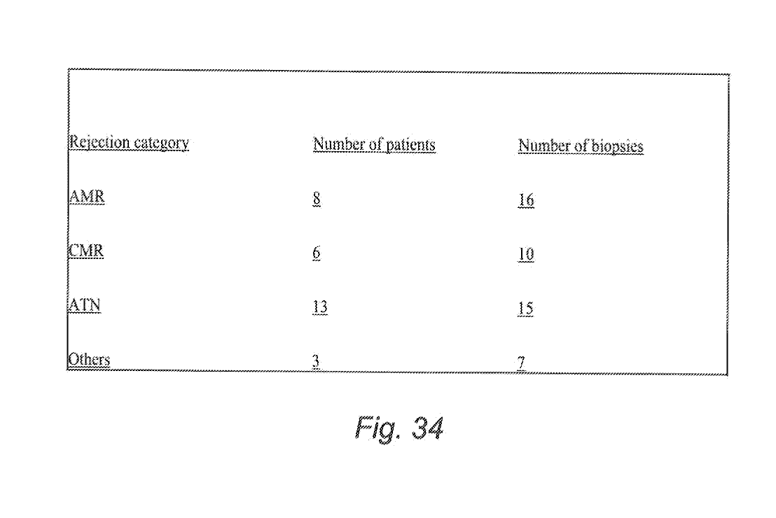

The experiment shown in FIG. 29 was performed to determine if NK cell activation in the CFC was via ADCC. Whole blood obtained from 5 each of individuals with CFC(+) and CFC(−) was used for the study. In the next experiment, immune cell numbers in a total of 48 HS living-donor kidney patients (31 campath 1H and 17 zenapax induction) transplanted after desensitization with IVIG and rituximab were measured pre-, post-desensitization and post-transplant by flow cytometry. Additionally, CFC against donor PBMCs (anti-donor CFC) and donor-specific antibodies (DSA) were monitored post-Tx in 37 HS living-donor kidney transplant patients desensitized with IVIG and rituximab, and the results were compared with biopsy findings (AMR, CMR and ATN). Finally in the Allo-CFC Assay, Whole blood (or blood cells in Study 1, responder) was incubated with irradiated peripheral blood mononuclear cells (PBMCs stimulator) and brefeldin A over night. IFNγ+ cells in CD3− cells were enumerated and results were expressed as a ratio against IFNγ+ cells without stimulation. A ratio >1 represents (+) response, and >5 represents high(+) response that might be associated with AMR.

### Example 35

### Methods—NK Cell Activation

To determine if NK cell activation in CFC is ADCC, plasma (p) was separated from whole blood of 5 HS w/CFC(+) (HSp) or 5 normal controls w/CFC(−) (NCp). Blood cells from HS (HSc) or NC (NCc) (responders) were incubated with irradiated peripheral blood mononuclear cells (PBMCs) pretreated with Sp or NCp (stimulators). IFNγ+/CD3− cells were enumerated and results expressed as a ratio vs. unstimulated cells. In addition, CFC against donor PBMCs, donor-specific antibodies (DSA) and cell subset numbers were monitored post-Tx in 37 ITS living-donor kidney Tx desensitized with intravenous immunoglobulin and rituximab, and induced w/Campath 1H (Camp) or Zenapax® (Zen).

### Example 36

### Results

Both HSc and NCc showed high(+) CFC against PBMCs pretreated with HSp, but not NCp (HSc: 6.1±2.3 vs. 1.4±0.9, p<0.01; NCc: 7.2±4.1 vs. 1.2±0.4, p<0.03), suggesting NK cell activation via ADCC. While not wishing to be bound by one particular theory, this implies that NK cells may contribute to AMR via ADCC without C4d deposition. Of 37 HS, 5 were diagnosed with ATN w/C4d(−). All had CFC(+) at biopsy with 2/5 DSA(+). 1/2 HS with CMR was CFC(+) with DSA(+). DSA at these episodes varied in level (>105-104 SFI). In the 1st 3 months post-Tx when CD19+ (0.12±0.21) (pre-desensitization 1.0), CD4+ (0.07±0.15) and CD8+ (0.13±0.20) cells were nearly undetectable in HS with Camp, NK cell# were 0.62±0.33 or 0.45±0.18 in HS with Camp or Zen, respectively.

The results of this study indicate: 1) NK cell activation against allo-Ag in CFC occurs primarily via ADCC, 2) NK cells are prevalent in HS post-Tx depleted of B/T cells, 3) NK cells activated through ADCC mechanisms may contribute to AMR, ATN and CMR in the HS as anti-donor CFC(+) and DSA(+) at various levels were often found at these episodes, and 4) NK activation may occur with low DSA or other antibodies.

### Example 37

### Conclusions

The inventors determined that NK cell activation against allo-Ag in CFC occurs primarily via ADCC. It was also determined that NK cells are prevalent in HS post-transplant depleted of B cells and T cells. Further, NK cells activated via ADCC mechanisms may contribute to AMR, ATN and CMR in the inventor's HS as anti-donor CFC(+) and DSA(+) at various levels were often found at these episodes. NK activation may occur with low DSA or other antibodies than HLA antibodies.

### Example 38

### Genes Responsible Antibody-Dependent Cell Cytotoxicity (ADCC) are Expressed in Kidney Biopsies from Highly Sensitized Patients (HS) with Antibody Mediated Rejection (AMR), Cell Mediated Rejection (CMR) and Acute Tubular Necrosis (ATN)

The inventors investigated the role of NK cells, especially via ADCC in AMR in HS patients, and identified ADCC-related genes in vitro using the whole genome approach. In addition, the expression of selected ADCC-related genes was analyzed in renal cortex tissue obtained from HS patients who had AMR and other kidney injuries.

### Example 39

### Methods

In the procedure for blood sample preparation, plasma was separated from whole blood of 2 HS with positive anti-allo-CFC results and 2 normal controls (NC) with negative anti-alloCFC results to produce the responder cells. The purpose of the plasma removal was to remove the anti-HLA antibodies in the HS blood. 3rd-party peripheral blood mononuclear cells (PBMCs) were irradiated and incubated in sera from HS patients or from NC. Blood cells from HS and NC (responders) were incubated for 3 hrs with irradiated PBMCs (stimulators) pretreated with HS sera (anti-HLA Ab+) mimicking the ADCC condition. Blood cells from HS and NC (responders) were incubated with PBMCs pretreated with NC plasma (anti-HLA Ab−) produced condition similar to MLR condition. HS cells and NC cells incubated without 3rd pary PBMCs served as negative controls. After 3 h incubation RNA was extracted from leukocytes using QIAamp RNA blood mini kit (Qiagen) and the quality of RNA was checked using Bioanalyzer (Agilent). RNA was amplified, labeled and hybridized on Affymetrix GeneChip Human Gene 1.0 ST arrays. After RMA normalization pairwise comparison between ADCC vs MLR, Control vs ADCC and Control vs MLR groups was performed using paired t-test.

In the procedure for kidney sample preparation, a total of 48 biopsies and 20 minimal-change control biopsies (MCD) were analyzed for 3 ADCC-related genes (IFNG, chemokines CCL3 and CCL4) and glyceraldehyde-3-phosphate dehydrogenase (GAPDH) as a control using reverse transcriptase real time-PCR (RT-PCR). RNA was extracted using RNeasy Micro kit (Qiagen) to ensure maximum RNA yield from small sample amounts. cDNA was synthesized using High Capacity cDNA Reverse Transcription Kit with RNase Inhibitor (Applied Biosystems, Foster City, Calif.). The gene expression was analyzed using the 7500 Real-Time PCR system and TaqMan® Gene Expression Assays for the selected genes (Applied Biosystems). The performance of the PCR was confirmed with the amplification of the housekeeping gene. Qualitative analysis (present or not present) was done using Fisher's exact test between the control and rejection groups.

### Example 40

### Results

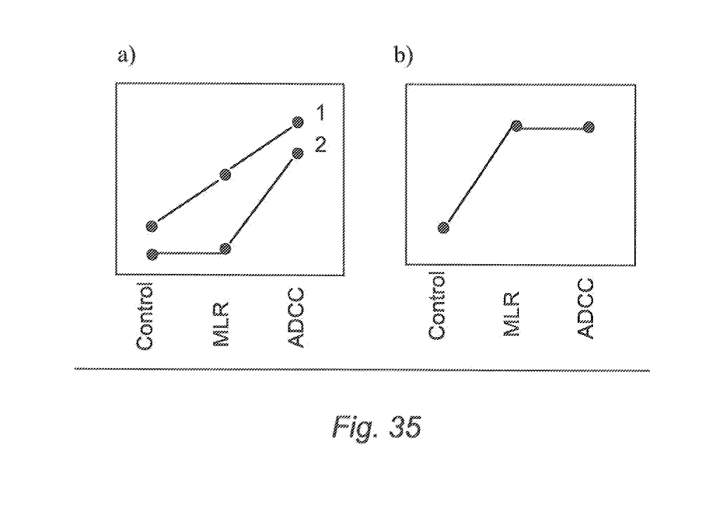

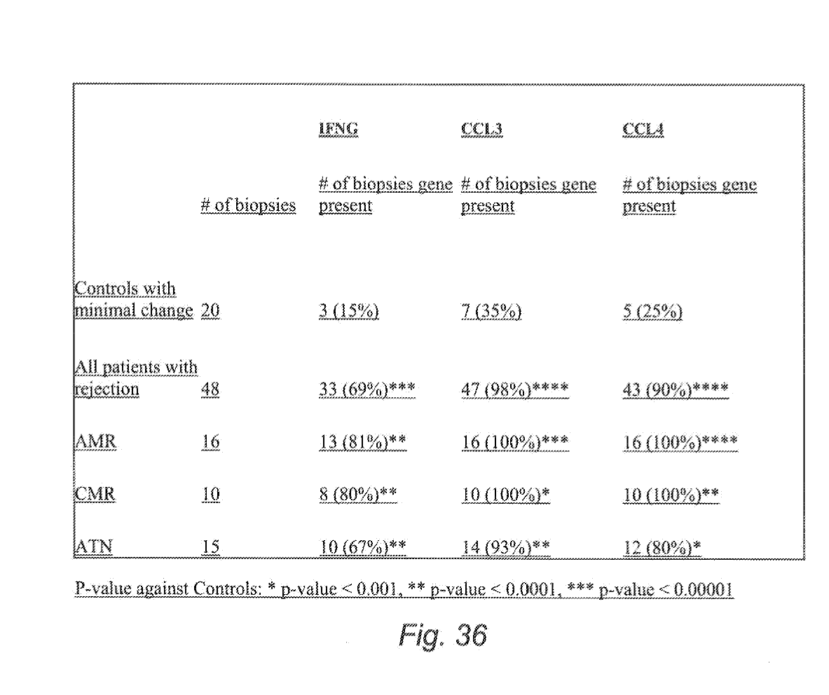

Pairwise comparison between ADCC vs. MLR, ADCC vs Control and MLR vs. Control was performed. FIG. 35 shows the ADCC-specific and MLR-specific gene expression patterns between 3 different conditions that the inventors aimed to identify. 12 ADCC-related genes were identified with >1.5-fold difference compared to MLR and Control (p<0.05), and an additional 46 transcripts with >1.5-fold expression in ADCC and MLR compared to Control were identified (p<0.05).

With respect to the biopsy results, GAPDH was detected in all biopsies. The expression of IFNG, CCL3 and CCL4 genes was significantly more in HS biopsies compared to MCD (IFNG: 33/48 vs. 3/20, p<0.001; CCL3: 47/48 vs. 7/20, p<0.001; CCL4: 43/48 vs. 5/20, p<0.001). Of interest was the high rate of expression of these genes (IFNG, CCL3 and CCL4) in biopsies obtained from HS w/AMR (13/16, 16/16, 16/16), CMR (8/10, 10/10, 10/10) and ATN (10/15, 14/15, 12/15, respectively).

### Example 42

### Conclusions

A total of 12 ADCC-related genes were identified by microarray analysis. Interestingly, many of the genes determined ADCC-specific were chemokine related, which are known to be important in NK cell function. Of 3 ADCC-related genes tested, most were expressed in biopsy tissue obtained from HS but were absent in MCD biopsies. These data indicate an important role for ADCC in mediation of injury to allografts in HS.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

[1] Jordan S C, et al. Evaluation of intravenous immunoglobulin as an agent to lower allosensitization and improve transplantation in highly sensitized adult patients with end-stage renal disease: report of the NIH IG02 trial. J Am Soc Nephrol. 2004; 15(12):3256-62.
[2] Jordan S C, et al. Intravenous gammaglobulin (IVIG): a novel approach to improve transplant rates and outcomes in highly HLA-sensitized patients. Am J Transplant. 2006; 6(3):459-66.
[3] Glotz D, et al. Desensitization and subsequent kidney transplantation of patients using intravenous immunoglobulins (IVIg). Am J Transplant. 2002; 2(8):758-60.
[4] Montgomery R A, et al. Plasmapheresis and intravenous immune globulin provides effective rescue therapy for refractory humoral rejection and allows kidneys to be successfully transplanted into cross-match-positive recipients. Transplantation. 2000; 70(6):887-95.
[5] Gloor J M, et al. Persistence of low levels of alloantibody after desensitization in crossmatch-positive living-donor kidney transplantation. Transplantation. 2004; 78(2):221-7.
[6] Vo A A, et al. Rituximab and intravenous immune globulin for desensitization during renal transplantation. N Engl J Med. 2008; 359(3):242-51.
[7] Vo A A, et al. Effect of induction therapy protocols on transplant outcomes in crossmatch positive renal allograft recipients desensitized with IVIG. Am J Transplant. 2006; 6(10):2384-90.
[8] Akalin E, Bromberg J S. Intravenous immunoglobulin induction treatment in flow cytometry cross-match-positive kidney transplant recipients. Hum Immunol. 2005; 66(4):359-63.
[9] Terasaki P I, Cai J. Humoral theory of transplantation: further evidence. Curr Opin Immunol. 2005; 17(5):541-5.
[10] Pescovitz M D. B cells: a rational target in alloantibody-mediated solid organ transplantation rejection. Clin Transplant. 2006; 20(1):48-54.
[11] Martin L, et al. Detection of donor-specific anti-HLA antibodies with flow cytometry in eluates and sera from renal transplant recipients with chronic allograft nephropathy. Transplantation. 2003; 76(2):395-400.
[12] Takahashi K. A new concept of accommodation in ABO-incompatible kidney transplantation. Clin Transplant. 2005; 19 Suppl 14:76-85.
[13] Reinsmoen N L, et al. Acceptable donor-specific antibody levels allowing for successful deceased and living donor kidney transplantation after desensitization therapy. Transplantation. 2008; 86(6):820-5.
[14] van Kampen C A, et al, Hoitsma A J, Allebes W A, Claas F H. Activated HLA class I-reactive cytotoxic T lymphocytes associated with a positive historical crossmatch predict early graft failure. Transplantation. 2002; 74(8):1114-9.
[15] Mulder A, et al. Determination of the frequency of HLA antibody secreting B-lymphocytes in alloantigen sensitized individuals. Clin Exp Immunol. 2001; 124(1):9-15. PMCID: 1906029.
[16] Mulder A, et al. Identification, isolation, and culture of HLA-A2-specific B lymphocytes using MHC class I tetramers. J Immunol. 2003; 171(12):6599-603.
[17] Zachary A A, et al. HLA-specific B cells: I. A method for their detection, quantification, and isolation using HLA tetramers. Transplantation. 2007; 83(7):982-8.
[18] Poggio E D, et al. Pretransplant cellular alloimmunity as assessed by a panel of reactive T cells assay correlates with acute renal graft rejection. Transplantation. 2007; 83(7):847-52.
[19] Korin Y D, et al. A novel flow assay for the detection of cytokine secreting alloreactive T cells: application to immune monitoring. Hum Immunol. 2005; 66(11):1110-24.
[20] Fredrich R, et al. The clinical significance of antibodies to human vascular endothelial cells after cardiac transplantation. Transplantation. 1999; 67(3):385-91.
[21] Jordan S C, et al. Hyperacute allograft rejection mediated by anti-vascular endothelial cell antibodies with a negative monocyte crossmatch. Transplantation. 1988; 46(4):585-7.
[22] Ismail A M, et al. Pretransplant detection of anti-endothelial cell antibodies could predict renal allograft outcome. Exp Clin Transplant. 2009; 7(2):104-9.
[23] Yard B, et al. The clinical significance of allospecific antibodies against endothelial cells detected with an antibody-dependent cellular cytotoxicity assay for vascular rejection and graft loss after renal transplantation. Transplantation. 1993; 55(6):1287-93.
[24] Wehner J, et al. Antibody and complement in transplant vasculopathy. Circ Res. 2007; 100(2): 191-203.
[25] Zhang X, Reed E F. Effect of Antibodies on Endothelium. Am J Transplant. 2009.
[26] Cerilli J, et al. The vascular endothelial cell antigen system. Transplantation. 1985; 39(3):286-9.
[27] Opelz G. Non-HLA transplantation immunity revealed by lymphocytotoxic antibodies. Lancet. 2005; 365(9470):1570-6.
[28] Lucchiari N, et al. Antibodies eluted from acutely rejected renal allografts bind to and activate human endothelial cells. Hum Immunol. 2000; 61(5):518-27.
[29] Glotz D, et al. Endothelial cells as targets of allograft rejection. Transplantation. 2006; 82(1 Suppl):519-21.
[30] Yokoyama W M, Plougastel B F. Immune functions encoded by the natural killer gene complex. Nat Rev Immunol. 2003; 3(4):304-16.
[31] Beilke J N, Gill R G. Frontiers in nephrology: the varied faces of natural killer cells in transplantation—contributions to both allograft immunity and tolerance. J Am Soc Nephrol. 2007; 18(8):2262-7.
[32] O'Leary J G, et al. T cell- and B cell-independent adaptive immunity mediated by natural killer cells. Nat Immunol. 2006; 7(5):507-16.
[33] LaRosa D F, et al. The innate immune system in allograft rejection and tolerance. J Immunol. 2007; 178(12):7503-9.
[34] Cooper M A, et al. The biology of human natural killer-cell subsets. Trends Immunol. 2001; 22(11):633-40.
[35] Koene H R, et al. Fc gammaRIIIa-158V/F polymorphism influences the binding of IgG by natural killer cell Fc gammaRIIIa, independently of the Fc gammaRIIIa-48L/R/H phenotype. Blood. 1997; 90(3):1109-14.
[36] Cartron G, et al. Therapeutic activity of humanized anti-CD20 monoclonal antibody and polymorphism in IgG Fc receptor FcgammaRIIIa gene. Blood. 2002; 99(3):754-8.
[37] Bowles J A, et al. Anti-CD20 monoclonal antibody with enhanced affinity for CD16 activates NK cells at lower concentrations and more effectively than rituximab. Blood. 2006; 108(8):2648-54. PMCID: 1895597.
[38] Toyoda M, et al. Correlation of cytomegalovirus DNA levels with response to antiviral therapy in cardiac and renal allograft recipients. Transplantation. 1997; 63(7):957-63.
[39] Radha R, et al. Cellular immune responses to cytomegalovirus in renal transplant recipients. Am J Transplant. 2005; 5(1):110-7.
[40] Toyoda M, et al Inhibition of allospecific responses in the mixed lymphocyte reaction by pooled human gamma-globulin. Transpl Immunol. 1994; 2(4):337-41.
[41] Breimer M E, et al. Multicenter evaluation of a novel endothelial cell crossmatch test in kidney transplantation. Transplantation. 2009; 87(4):549-56.
[42] Kim D H, et al. FCGR3A gene polymorphisms may correlate with response to frontline R-CHOP therapy for diffuse large B-cell lymphoma. Blood. 2006; 108(8):2720-5.
[43] Rifle G, Mousson C, Martin L, Guignier F, Hajji K. Donor-specific antibodies in allograft rejection: clinical and experimental data. Transplantation 2005; 79(3 Suppl):S14-8.
[44] Toyoda M, Pao A, Vo A, Lukovsky M, Radha R, Sado T, et al. Intracellular IFNgamma production in CD3 negative cells exposed to allo-antigens is an indicator of prior sensitization. Transpl Immunol 2010; 22:121-7.
[45] van Kampen C A, Versteeg-vd Voort Maarschalk M F, Langerak-Langerak J, Roelen D L, Claas F H. Kinetics of the pregnancy-induced humoral and cellular immune response against the paternal HLA class I antigens of the child. Hum Immunol 2002; 63(6):452-8.
[46] Van Rood J J, Eernisse J G, Van Leeuwen A. Leucocyte antibodies in sera from pregnant women. Nature 1958; 181(4625):1735-6.
[47] Suciu-Foca N, Reed E, Rohowsky C, Kung P, King D W. Anti-idiotypic antibodies to anti-HLA receptors induced by pregnancy. Proc Natl Acad Sci USA 1983; 80(3): 830-4.
[48] Reed A, Pirsch J, Armbrust M J, Burlingham W J, Knechtle S J, D'Alessandro A M, et al. Multivariate analysis of donor-specific versus random transfusion protocols in haploidentical living-related transplants. Transplantation 1991; 51(2):382-4.
[49] Qian Z, Lee C Y, Murata K, Liu J, Fox-Talbot K, Wasowska B A, et al. Antibody and complement mediated injury in transplants following sensitization by allogeneic blood transfusion. Transplantation 2006; 82(7):857-64.
[50] Terasaki P I, Cecka J M, Gjertson D W, Takemoto S. High survival rates of kidney transplants from spousal and living unrelated donors. N Engl J Med 1995; 333(6): 333-6.
[51] Pollack M S, Trimarchi H M, Riley D J, Casperson P R, Manyari L E, Suki W N. Shared cadaver donor-husband HLA class I mismatches as a risk factor for renal graft rejection in previously pregnant women. Hum Immunol 1999; 60(11):1150-5.
[52] Rosenberg J C, Jones B, Oh H. Accelerated rejection following offspring-to-mother and husband-to-wife transplants. Clin Transpl 2004; 18(6):729-33.
[53] van den Boogaardt D E, van Rood J J, Roelen D L, Claas F H. The influence of inherited and noninherited parental antigens on outcome after transplantation. Transpl Int 2006; 19(5):360-71.
[54] van Kampen C A, Versteeg-van der Voort Maarschalk M F, Langerak-Langerak J, van Beelen E, Roelen D L, Claas F H. Pregnancy can induce long-persisting primed CTLs specific for inherited paternal HLA antigens. Hum Immunol 2001; 62(3):201-7.
[55] El-Awar N R, Akaza T, Terasaki P I, Nguyen A. Human leukocyte antigen class I epitopes: update to 103 total epitopes, including the C locus. Transplantation 2007; 84(4):532-40.
[56] El-Awar N, Lee J H, Tarsitani C, Terasaki P I. HLA class I epitopes: recognition of binding sites by mAbs or eluted alloantibody confirmed with single recombinant antigens. Hum Immunol 2007; 68(3):170-80.
Transplantation 2002; 74(8):1114-9.
[57] Sun J C, Beilke J N, Lanier L L. Adaptive immune features of natural killer cells. Nature 2009; 457(7229):557-61 PMCID: 2674434.
application to immune monitoring. Hum Immunol 2005; 66(11):1110-24.
[58] Dankers M K, Roelen D L, Korfage N, de Lange P, Witvliet M, Sandkuijl L, et al. Differential immunogenicity of paternal HLA class I antigens in pregnant women. Hum Immunol 2003; 64(6):600-6.

1. A method of diagnosing susceptibility to a condition caused by any mechanism involving rejection mediated by an antibody (RMA), in an individual, comprising:
obtaining a biological sample from the individual;
assaying the sample to determine the presence or absence of an abnormal level of expression, relative to an individual who does not have ADCC in a kidney allograft or RMA, of one or more of the genes described in FIG. 17 and/or FIG. 37 herein; and
diagnosing susceptibility to the condition based upon the presence of an abnormal level of expression, relative to an individual who does not have ADCC in the kidney allograft or RMA, of one or more genes described in FIG. 17 and/or FIG. 37 herein.

2. The method of claim 1, wherein susceptibility is determined prior to and/or after a subject receives an organ transplant

3. A method of diagnosing susceptibility to a condition caused by any mechanism involving rejection mediated by an antibody (RMA) in an individual, comprising:
obtaining a biological sample from the individual;
assaying the sample to determine the presence or absence of an elevated allo-antigen response; and
diagnosing susceptibility to RMA based upon the presence of an elevated allo-antigen response.

4. The method of claim 3, wherein assaying the sample comprises using cytokine flow cytometry to determine an allo-antigen response to peripheral blood mononuclear cells (PBMC) and/or endothelial cells (EC) and/or flow or luminex beads coated with natural or recombinant HLA antigens.

5. The method of claim 4, wherein the allo-antigen response is determined by the detection of IFNγ producing cells.

6. The method of claim 4, wherein the sample comprises Natural Killer (NK) cells.

7. The method of claim 4, wherein the sample comprises CD3− cells.

8. The method of claim 5, wherein the IFNγ producing cells are CD3− cells

9. The method of claim 3, wherein the biological sample comprises: biopsied tissue, blood, sera, plasma, or combinations thereof.

10. The method of claim 3, wherein the biological sample comprises biopsied kidney tissue.

11. The method of claim 3, wherein the individual is a female with a history of pregnancy.

12. The method of claim 4, wherein the PBMC and/or EC are derived from a prospective organ donor and/or one or more non-donors.

13. The method of claim 3, further comprising, determining the FCγRIIIa genotype of the individual,
wherein if the individual has negative or low anti-allo reactivity and a FCγRIIIa-FF genotype then the susceptibility to the condition is a relatively low risk of developing the condition, and
wherein if the individual is positive for one allo-CFC and has a genotype of FcγRIIIa-FF or VF then susceptibility to the condition is a relatively moderate risk of developing the condition, and
wherein if the individual is positive for at least one allo-CFC and has a FcγRIIIa-VV genotype then the susceptibility to the condition is a relatively high risk of developing the condition.

14. A method of transplanting an organ to an individual, comprising:
diagnosing a lack of susceptibility to a condition caused by any mechanism involving rejection mediated by an antibody (RMA) according to the method of claim 4 in the individual; and
transplanting the organ to the individual.

15. The method of claim 14, wherein the organ is a kidney.

16. The method of claim 14, wherein the organ comprises: a whole organ, a portion of an organ, skin, or other tissue or tissues suitable for transplant.
